(19) United States
(12) Patent Application Publication
(10) Pub. No.: US 2023/0371900 A1
(43) Pub. Date: Nov. 23, 2023

(54) AUTOMATED OPERABILITY AND NAVIGATION OF AUTONOMOUS SMART MEDICAL DEVICES

(71) Applicant: Bruce REINER, Berlin, MD (US)

(72) Inventor: Bruce REINER, Berlin, MD (US)

(21) Appl. No.: 18/362,616

(22) Filed: Jul. 31, 2023

Related U.S. Application Data
(63) Continuation-in-part of application No. 17/836,742, filed on Jun. 9, 2022.
Application No. 17/836,742 is a continuation-in-part of application No. 17/712,693, filed on Apr. 4, 2022.
Application No. 17/712,693 is a continuation of application No. 16/503,920, filed on Jul. 5, 2019, now Pat. No. 11,324,451.
Application No. 16/503,920 is a continuation-in-part of application No. 15/632,817, filed on Jun. 26, 2017, now abandoned.
Application No. 17/836,742 is a continuation-in-part of application No. 17/575,048, filed on Jan. 13, 2022.
Application No. 17/575,048 is a continuation of application No. 15/434,783, filed on Feb. 16, 2017, now Pat. No. 11,224,382.
(60) Provisional application No. 62/694,248, filed on Jul. 5, 2018.
(60) Provisional application No. 62/355,031, filed on Jun. 27, 2016.
(60) Provisional application No. 62/295,787, filed on Feb. 16, 2016.
(60) Provisional application No. 63/422,616, filed on Nov. 4, 2022.
(60) Provisional application No. 63/394,823, filed on Aug. 3, 2022.

(51) Int. Cl. A61B 5/00; A61B 5/07; A61B 34/30

(52) U.S. Cl. CPC A61B 5/6861; A61B 5/07; A61B 34/30; A61B 2090/3983

(57) ABSTRACT

The present invention relates to autonomous medical devices, which are capable of self-navigation with real-time adjustment for changing anatomy and pathology. The autonomous medical devices include embedded signal emitters and/or receivers, which perform real-time tracking, and which create real-time anatomic visualization maps for the purposes of monitoring smart device activity and location in vivo, to ensure proper localization of the devices in question, and augment guidance technologies contained within the medical devices. The data derived from the smart medical device technologies can be automatically recorded, stored, and analyzed for the purpose of determining best practices, and the creation of machine learning and artificial intelligence algorithms. The autonomous smart medical devices can be applied to a wide variety of medical applications and work in combination with one another in the performance of complex medical tasks to create independent medical technology which can rapidly adapt, iteratively learn, and synergistically function in vivo.

### CROSS-REFERENCE TO RELATED APPLICATIONS

The present invention claims priority from U.S. Provisional Patent Application Nos. 63/422,616 filed Nov. 4, 2022, and 63/394,823 filed Aug. 3, 2022, and is a Continuation-in-Part (CIP) of U.S. Nonprovisional patent application Ser. No. 17/836,742 filed Jun. 9, 2022, U.S. Nonprovisional patent application Ser. No. 17/712,693 filed Apr. 4, 2022, and U.S. Nonprovisional Ser. No. 17/575,048 filed Jan. 13, 2022, the contents of all of which are herein incorporated by reference in their entirety.

### BACKGROUND OF THE INVENTION

### 1. Field of the Invention

The present invention relates to autonomous medical devices, which are capable of self-navigation with real-time adjustment for changing anatomy and pathology.

### 2. Description of the Related Art

In conventional medical practice, medical devices are manually inserted and navigated under the direct control of a skilled medical professional. Depending upon the specific type of device and the desired anatomic positioning, this placement may be performed with or without directional assistance. In the case where no ancillary assistance is provided, the device is positioned blindly, based entirely on the technical skill, education, and practical experience of the operator. In other cases, external visualization guidance is provided to the operator, which typically takes the form of conventional medical imaging technologies (e.g., radiography, fluoroscopy) which provide visual guidance during the course of the medical device placement. Regardless of the technique employed, once the medical device positioning has been completed, medical imaging is often performed to verify final device positioning, before the device is actively used.

In accordance with existing medical device technology, a number of physical constraints limit the adaptability and evolution of in vivo medical devices. These constraints are tied to a variety of limiting factors related to the operator, host subject, physical environment in which the device is deployed, required functionality of the device, as well as the methods used for device introduction and transport.

In current practice, in vivo medical devices are positioned into the host subject by either traditional invasive techniques (e.g., surgery) or percutaneously, using minimally invasive techniques (e.g., coronary artery catheterization). Minimally invasive device placement is generally preferred due to reduced patient morbidity and recovery time. The disadvantage of minimally invasive device placement is that it is both operator, patient, and technology dependent. When any one of these factors is deficient, the end result may be suboptimal.

For minimally invasive vascular catheter placement, the operator (which can be human or robotic), routinely makes a skin incision through which the device (e.g., catheter) and guidewire will be inserted. Guidewires are metallic wires which facilitate the passage of the catheter, which on its own, would be limited due to physical constraints. The components of guidewires include an inner core made of stainless steel or nitinol, an outer body made of coils or polymers, a distal flexible tip made of platinum or tungsten alloy, and a surface coating.

The passage of the vascular catheter is determined by two often opposing forces, pushability and navigation. Pushability refers to the force required to advance the catheter to its designated site, while navigation refers to the ability of the catheter to move freely through a non-linear pathway like the vascular system.

Navigation requires that the catheter shaft remains flexible in order to easily bend to accommodate to the curvature of the blood vessel in which it travels, without causing traumatic injury. Reducing catheter shaft diameter, wall thickness, and flexural modulus can improve catheter flexibility and navigation.

In order to advance the catheter, sufficient push force must be exerted by the operator to overcome the friction forces between the outer surface of the catheter shaft and the interior vessel wall. As the catheter advances and vascular surface contact increases, the push force must also increase in order to continue advancement of the catheter. As these push forces increase, the catheter shaft is prone to buckle and kink, which impedes successful placement. This tendency to kink can be addressed by a variety of structural modifications including increased catheter shaft diameter, wall thickness, and flexural modulus of the catheter shaft material (which is the ability of the material to bend). One can see that these forces of pushability and navigation often act in opposition to one another, creating challenges for minimally invasive device placement.

For devices introduced using currently available minimally invasive techniques, device maneuverability and steering capabilities are limited by torque and the frictional forces between the catheter and blood vessel walls, as defined by Euler-Bernoulli beam and Cosserat rod theories. Using conventional push-pull and twisting techniques, the operator attempts to maneuver the device, often incurring damage to the vessel.

A number of iatrogenic complications may occur during or after the placement of medical devices, which may be the result of direct tissue injury, device mispositioning, or device malfunctioning. Thus, it is common for underlying vascular tortuosity and/or obstruction to prevent successful navigation, even in the hands of an experienced and technically proficient operator.

Take for example placement of an endotracheal tube, which is inserted for the purpose of mechanical ventilation. During the insertion process, the endotracheal tube can physically damage the trachea or adjacent anatomic structures (e.g., vocal cords), requiring clinical intervention to address the resulting damage. At the same time, if the device is improperly positioned in the right mainstem bronchus, it will fail to ventilate the contralateral left lung, resulting in left lung atelectasis (i.e., collapse), requiring urgent repositioning of the endotracheal tune and respiratory therapy to re-expand the collapsed left lung. (In rare cases, if the collapsed left lung is not promptly re-expanded more severe damage can occur from diminished oxygenation (e.g., myocardial infarction, stroke). Lastly, when the inserted device is not properly functioning, the device requires removal and replacement with another functioning device. The resulting adverse outcome is in part dependent upon the severity and duration of the faulty device.

But even when a medical device is properly positioned and shown to be properly working, delayed complications may occur, some of which may be due to device positional change which goes undetected. In the example of the endotracheal tube, flexion of the patient's head may cause the initially properly positioned endotracheal tube to advance several centimeters, resulting in the improper positioning of the endotracheal tube in the right mainstem bronchus and left lung collapse. In reality, medical device migration and repositioning is a common occurrence in everyday clinical practice and may serve as a relatively common iatrogenic complication.

The frequency and severity of medical device iatrogenic complications often increase as the medical device becomes more specialized, is in a more distant and harder to reach anatomic location, is of longer duration, and whose functionality is dependent upon millimeter-specific positioning. As an example, even a minor positional change for a hepatic intravascular catheter tasked with selective chemotherapy infusion may result in damage to normal liver tissue and failure of the chemotherapeutic agent to reach the targeted malignant cells. As such, it is critical to ensure that the catheter is properly positioned at all times, and when even a minor positional change takes place, immediate corrective action is taken.

One way to address such a complication is routine medical imaging surveillance (e.g., daily x-rays), which may come at the cost of delayed diagnosis, increased cost of care, and excessive ionizing radiation (which adversely affects patient safety).

A number of technical developments in catheter design and construction have been created in an attempt to address these challenges. These include (but are not limited to) improvements in shaft materials (e.g., high-consistency silicone rubbers (HCR) and liquid silicone rubbers (LSR), reduction of vascular friction through hydrophilic catheter coatings, segmented catheter design, use of nano clays for polymer reinforcement, and creation of manually steerable catheters.

However, these advancements are ultimately constrained by the guidewire system and manual forces used for device transport. As long as these factors remain, the evolution of medical devices will remain in a relatively limited state.

Accordingly, in spite of these advancements, minimally invasive catheter placement remains problematic and as previously stated is often operator and patient dependent. Operator dependence is often dictated by the individual skills, expertise, and experience of the operator.

Patient dependence is often determined by patient clinical status, body habitus, and ability to follow commands. At the same time, a patient's underlying pathology (e.g., arterial occlusive disease) will often serve as a determining factor in procedural success or failure. Simply stated, when device placement involves inherent deficiencies in the operator and/or patient, success is far from guaranteed and may incur high rates of iatrogenic complication (e.g., bleeding, tissue injury).

Thus, the present invention provides an alternative approach to medical device placement and navigation, which is currently not available and addresses many of the existing pitfalls and challenges intrinsic to conventional practice.

### SUMMARY OF THE INVENTION

The present invention relates to autonomous medical devices, which are capable of self-navigation with real-time adjustment for changing anatomy and pathology.

The present invention accomplishes these goals in a variety of ways, including (but are not limited to) the creation of autonomous smart medical devices, which are capable of automated navigation and operability.

The autonomous medical devices include embedded signal emitters and/or receivers, which perform real-time tracking, and which create real-time anatomic visualization maps for the purposes of monitoring smart device activity and location in vivo, to ensure proper localization of the devices in question, and augment guidance technologies contained within the medical devices. The data derived from the smart medical device technologies can be automatically recorded, stored, and analyzed for the purpose of determining best practices, which can be applied to the creation of machine learning and artificial intelligence algorithms. The autonomous smart medical devices can be applied to a wide variety of medical applications and disciplines and work in combination with one another in the performance of complex medical tasks, to create independent medical technology which can rapidly adapt, iteratively learn, and synergistically function in vivo, with or without human operator input and guidance.

The ability of these smart medical devices to perform these highly specialized functions in vivo is in part dependent upon exact and accurate positioning of the smart medical device and its subcomponents on as little as a millimeter level. Since conventional methods for medical device placement do not routinely provide this degree of positional specificity, in order to facilitate such exact and accurate smart device in vivo positioning, one may require a detailed, accurate, and dynamic methodology for three-dimensional (3D) anatomic visualization. While a number of existing medical imaging technologies are currently in use for medical device placement and surveillance (e.g., radiography, fluoroscopy, computer tomography), they have a number of associated deficiencies including (but are not limited to) limitation in anatomic resolution, requirement for repeated data collection, safety concerns related to ionizing radiation and iatrogenic complications, and static nature of the derived data.

However, the present invention is a novel leap forward in improvement on the existing technology and provides for a novel method of automating smart medical device operability and navigation in vivo. The present invention creates a device navigational system which provides instantaneous and continuous feedback to authorized medical professionals, while proactively taking any necessary corrective action to ensure patient safety, proper device performance, and optimized clinical outcomes. The present invention accomplishes these goals through the creation of a dynamic medical device guidance and surveillance system, which can provide automated alerts in the event of improper device positioning and/or performance.

The present invention is an improvement on the existing technology and provides for a novel method of automating smart medical device operability and navigation in vivo. The net result is the creation of a synergistic process by which smart medical devices can be inserted within a given host, navigated to a specific position of anatomic and/or pathologic concern, and performance of a variety of diagnostic and/or therapeutic functions (on macroscopic and/or microscopic levels), with the goal of improving patient clinical outcomes.

In one important application of the present invention, the navigation of these smart medical devices can be completely automated, effectively creating an autonomous smart device, which is capable of self-navigation, deployment, and repositioning, in accordance with the specific clinical indication, host pathology, and device functionality. The ultimate goal of the present invention is the creation of smart devices which are intuitive, data-driven, interactive, self-corrective, and intelligent.

The net result is the creation of a synergistic process by which smart medical devices can be inserted within a given host, navigated to a specific position of anatomic and/or pathologic concern, and performance of a variety of diagnostic and/or therapeutic functions (on macroscopic and/or microscopic levels), with the goal of improving patient clinical outcomes.

The solution to these challenges is the creation of a device navigational system which provides instantaneous and continuous feedback to authorized medical professionals, while proactively taking any necessary corrective action to ensure patient safety, proper device performance, and optimized clinical outcomes. One way to accomplish these goals is through the creation of a dynamic medical device guidance and surveillance system, which can provide automated alerts in the event of improper device positioning and/or performance.

In one embodiment, a system which performs medical tasks in a body of a patient, includes: a medical device, including: a signal emitter which emits energy in a form of a transmitted signal; a signal receiver which receives transmitted energy as a received signal; a plurality of sensors and/or detectors; a propulsion mechanism and/or a steering mechanism; an energy source; and at least one processor which receives anatomic and positional data from the plurality of sensors and/or detectors and records the data in a database; wherein the medical device is inserted in the patient and collects the anatomic and positional data in real-time from the plurality of sensors and/or detectors; and wherein the at least one processor dynamically analyzes the anatomic and positional data on a continuous basis such that the medical device at least partially autonomously navigates to a desired position in the patient.

In one embodiment, the at least one processor is internal to the medical device, and further includes: an external signal receiver and/or transmitter which receives the transmitted signal from the medical device; at least one external controller which receives the transmitted signal from the external signal receiver and/or transmitter and converts the transmitted signal into a standardized form of data; and at least one external processor which receives the data from the external controller and records the data in a separate database.

In one embodiment, the propulsion system includes at least one of chemically powered motors, enzymatically powered motors, external field driven motors, internally mounted miniaturized electrodes, miniaturized electromagnetic pumps, or appendages.

In one embodiment, the system further includes: an external energy charging source; wherein the energy storage in the medical device can receive energy externally transmitted to the medical device from the external charging source; and wherein the energy source is one of batteries, biofuel cells, thermoelectricity, piezoelectric generators, photovoltaic cells, or ultrasonic transducers.

In one embodiment, the system further includes: an anchoring device attached to or disposed in the medical device, which anchors the medical device to the desired position.

In one embodiment the medical device further includes: a lidar scanner which detects physical surroundings and distances from the medical device; a plurality of inertial sensors which record movement of the medical device; and at least one camera which provides visual tracking information to the medical device.

In one embodiment, the medical device further includes: a gyroscope which measures or maintains orientation and angular velocity of the medical device; and a Global Positioning System (GPS) which provides the user with positioning, navigation and timing information of the medical device.

In one embodiment, the medical device further includes: a plurality of compartments containing at least one of: a spring-actuated device, including at least one of a cauterization tool or a cutting tool; a delivery device which delivers a product; or an ejection device which ejects a product.

In one embodiment, the processor utilizes artificial intelligence in the analysis of the anatomic and positional data.

In one embodiment, the medical device collects data from other medical devices in vivo and synchronizes the collected data in real-time for the processor to perform the analysis and produce at least one of a 3-D or 4-D anatomic visualization map which is used in at least the partial autonomous navigation of the medical device.

In one embodiment, the medical device under said at least partial autonomous navigation performs course corrections needed to stay on course to the desired position.

In one embodiment, the medical device deploys a marker from one of the plurality of compartments, the marker which emits signals processed by the processor of the medical device, which allows the medical device to position itself accurately at the desired position.

In one embodiment, the 3-D or 4-D anatomic visualization map is cross-correlated with at least one external visualization map.

In one embodiment, the data used in said 3-D or 4-D anatomic visualization map is used to edit at least one external anatomic dataset to provide an updated version thereof.

In one embodiment, the data used in the 3-D or 4-D anatomic visualization map is combined with a plurality of external datasets to produce a single all-inclusive anatomic visualization map.

In one embodiment, the anatomic and positional data collected by the medical device is provided to at least one other medical device by emitting signals from the signal emitter of the medical device, to facilitate autonomous navigation of the other medical devices to the desired position.

In one embodiment, the medical device includes a plurality of subcomponents attached to a main body, said plurality of subcomponents which can detach from the main body for individual navigation, and re-attach with the main body.

In one embodiment, the medical device is capable of merging with other medical devices and/or subcomponents into an aggregate medical device to increase functionality.

In one embodiment, the medical device is capable of at least one of collapsing in size by one of detaching one or more components or expanding in size by expanding on one or more components.

In one embodiment, the medical device is capable of being eliminated from the body of the patient or extracted from the body of the patient.

In one embodiment, the elimination occurs through gastrointestinal, urinary, respiratory or dermal systems; and extraction occurs through one of towing the medical device by the at least one other medical device or through surgery.

In one embodiment, autonomous navigation of the medical device is capable of being turned on or turned off.

In one embodiment, the medical device is capable of being extracted from the body of the patient; and extraction occurs through one of towing the medical device by the at least one other medical device or through surgery.

In one embodiment, the system further includes a mechanism for immediate intervention in an emergency, the mechanism which circumvents security protocols; and wherein a plurality of alerts is automatically transmitted by electronic methods to authorized parties.

In one embodiment, in the immediate intervention, the medical device is placed in a range of modes from semi-active to turned off.

In one embodiment, the medical device is turned off in accordance with a timer of variable duration.

In one embodiment, the medical device tracks said at least one other medical device and on condition that a movement of said at least one other medical device is contrary to programmed expectations, said medical device transmits a warning signal via electronic methods to a user and to other medical devices.

In one embodiment, all device communication between the medical device and at least one other medical device is recorded in the external database.

In one embodiment, the medical device is turned off automatically in at least one of: an absence of a corroborating signal from a partnering medical device, upon receipt of a distress signal from the partnering medical device, upon receipt of a signal from the external processor monitoring communications from the medical device, upon command from an authorized user monitoring the communications, upon activation of the mechanism for immediate intervention, upon cessation of activity due results of an audit and analysis of communications between the medical device and other medical devices, upon activation of intervention of other medical devices which act to minimize impact of a shutdown failure.

In one embodiment, the mechanism for immediate intervention includes self-destruction.

In one embodiment, a method of performing medical tasks in a body of a patient, includes: receiving a plurality of signals from a plurality of sensors and/or detectors disposed in at least one medical device at a processor of the at least one medical device; wherein the plurality of signals provide anatomic and positional data in real-time to the processor of the at least one medical device; emitting a plurality of signals to a plurality of other medical devices and/or to an external processor, the plurality of signals which provide the anatomic and positional data to said plurality of other medical devices and/or to the external processor; wherein the at least one processor dynamically analyzes the anatomic and positional data on a continuous basis such that the at least one medical device at least partially autonomously navigates to a desired position in the patient.

Thus, has been outlined, some features consistent with the present invention in order that the detailed description thereof that follows may be better understood, and in order that the present contribution to the art may be better appreciated. There are, of course, additional features consistent with the present invention that will be described below, and which will form the subject matter of the claims appended hereto.

In this respect, before explaining at least one embodiment consistent with the present invention in detail, it is to be understood that the invention is not limited in its application to the details of construction and to the arrangements of the components set forth in the following description or illustrated in the drawings. Methods and apparatuses consistent with the present invention are capable of other embodiments and of being practiced and carried out in various ways. Also, it is to be understood that the phraseology and terminology employed herein, as well as the abstract included below, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the methods and apparatuses consistent with the present invention.

### BRIEF DESCRIPTION OF THE DRAWINGS

The description of the drawings includes exemplary embodiments of the disclosure and are not to be considered as limiting in scope.

FIG. 1 shows a schematic drawing of internal and external views of an (automated) smart medical device and system having an assortment of miniaturized components that perform navigation and medical tasks, according to one embodiment consistent with the present invention.

FIG. 2 shows a schematic drawing of internal and external views of an (automated) smart medical device and system according to one embodiment consistent with the present invention. The device shows an assortment of miniaturized components that can perform various tasks in the body using the same platforms (i.e., spring device, ejection device etc.), according to embodiments consistent with the present invention.

FIG. 3 shows a schematic drawing of an (automated) smart medical device such as a catheter having an assortment of miniaturized components that perform navigation and medical tasks, according to one embodiment consistent with the present invention.

FIG. 4 shows a plan view of an (automated) smart medical device with a plurality of component smart devices attached to the body of the smart medical device, and shows the ability of the component smart devices to compress/decompress or detach/re-attach from the smart medical device main body, according to one embodiment consistent with the present invention.

FIG. 5 shows a plan view of an (automated) smart medical device with the ability of one section of the smart medical device able to detach/re-attach from the main body and function separately if desired, according to one embodiment consistent with the present invention.

FIG. 6 shows a plan view of an (automated) smart medical device with the ability of one or more sections able to detach/re-attach into separate working components, according to one embodiment consistent with the present invention.

FIGS. 7A and 7B show flow charts of an operation of an autonomous medical device, capable of self-navigation with real-time adjustment for changing anatomy and pathology, according to one embodiment consistent with the present invention.

### DESCRIPTION OF THE INVENTION

The present invention relates to autonomous medical devices, which are capable of self-navigation with real-time adjustment for changing anatomy and pathology. The autonomous medical devices include embedded signal emitters and/or receivers, which perform real-time tracking, and which create real-time anatomic visualization maps for the purposes of monitoring smart device activity and location in vivo, to ensure proper localization of the devices in question, and augment guidance technologies contained within the medical devices. The data derived from the smart medical device technologies can be automatically recorded, stored, and analyzed for the purpose of determining best practices, which can be applied to the creation of machine learning and artificial intelligence algorithms. The autonomous smart medical devices can be applied to a wide variety of medical applications and disciplines and work in combination with one another in the performance of complex medical tasks, to create independent medical technology which can rapidly adapt, iteratively learn, and synergistically function in vivo, with or without human operator input and guidance.

Autonomous medical devices or vehicles rely on a combination of sensors, actuators, complex algorithms, machine learning systems, and powerful computer-based processors to execute self-navigation software. A combination of radar, LIDAR (“light detection and ranging” or “laser imaging, detection, and ranging”), and ultrasonic sensors serve to monitor position of nearby devices, measure distances, and detect obstructions. Video cameras serve to show device placement and obstructions during travel. In turn, the software processes sensory inputs, plots a path, sends instructions to actuators, and controls acceleration, movement and steering.

While the autonomous smart medical devices of the present invention may share some of the same components of other self-navigational devices, the present medical goals require substantive technical differences. Whereas other autonomous vehicles navigate in large open space (i.e., drones), smart medical devices often navigate in confined quarters such as blood vessels and lymphatics, whose diameter measurements are on the order of centimeters (cms) and millimeters (mms). As a result, the navigational requirements for in vivo smart medical devices are more onerous and exact.

In the present inventor's previous U.S. patent application Ser. No. 17/712,693, filed Apr. 4, 2022, and U.S. patent application Ser. Nos. 17/575,048 and 17/836,742, filed Jun. 9, 2022, and Jan. 13, 2022, respectively, all of which are incorporated herein by reference (hereinafter the “incorporated patents/applications”), an apparatus and methodology were described for the creation for in vivo smart medical devices with embedded biosensors and miniaturized devices which provide new and novel applications for both diagnostic and therapeutic medical applications.

The present invention expands the functionality of those smart medical devices into new and novel areas, including (but are not limited to) targeted cellular and tissue collection, real-time and dynamic data collection and analysis of physiology, microscopic and macroscopic localization of pathology, microsurgery, targeted drug delivery, thermal ablation, cryotherapy, stem cell implantation, embolization, atherectomy, and cauterization.

In addition, the medical applications of nanobots and their corresponding energy supplies were described in detail in the inventor's prior patents and applications, namely U.S. Pat. Nos. 11,224,382, 11,324,451, and U.S. patent application Ser. No. 17/836,742, filed Jun. 9, 2022, all of which are herein incorporated by reference in their entirety (hereinafter included in the “incorporated patents/applications”). Further, an alternative strategy for anatomic visualization was described in the inventor's previous U.S. patent application Ser. No. 17/836,742, filed Jun. 9, 2022, and U.S. patent application Ser. No. 17/712,693 filed Apr. 4, 2022, all of which are herein incorporated by reference in their entirety (hereinafter included in the “incorporated patents/applications”).

The above referenced pending patent applications and their priority patents/applications (i.e., the “incorporated patents/patent applications”) describe the creation of medical device technology capable of fully autonomous in vivo self-navigation with the ability to perform real-time dynamic adjustment and adaptability to ever changing physiologic, anatomic, and pathologic conditions within the host subject.

The present invention relates to a system in which smart medical devices can operate in vivo in both assisted and non-assisted manners, for extended periods of time, in a self-directed and highly intelligent manner, in response to changing anatomic and pathologic states. These are novel functional and technical capabilities, which do not currently exist in medical practice and its supporting technologies.

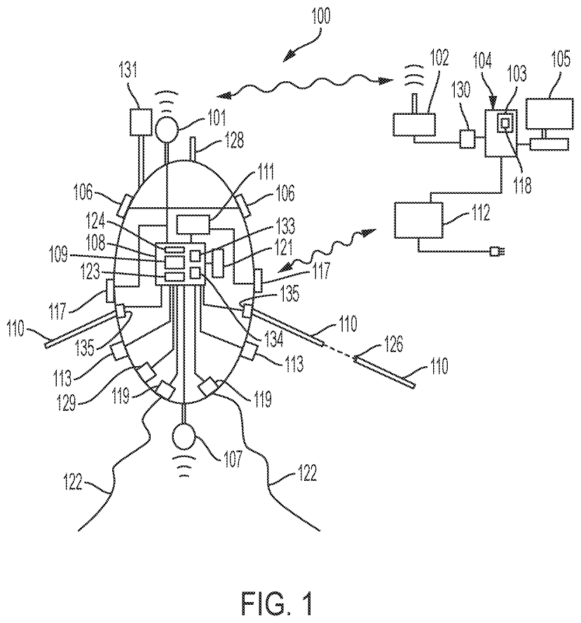

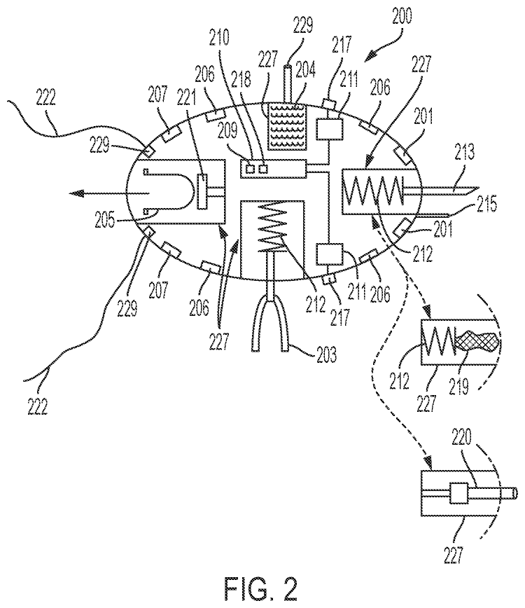

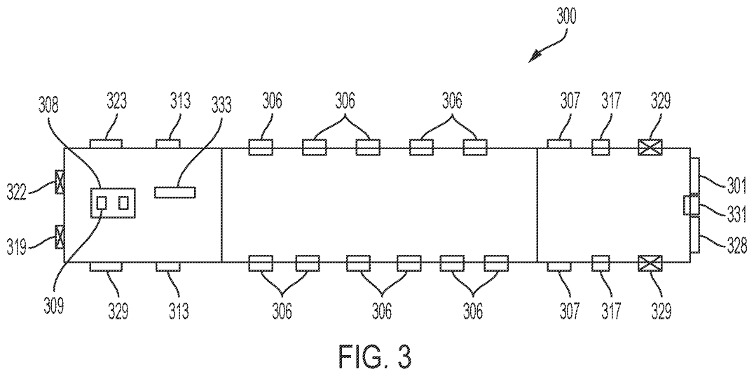

In one embodiment, a wide array of devices and sensors can be embedded within smart medical devices (see FIGS. 1-3, for example), as discussed in detail in the incorporated patents/patent applications, with each smart device having certain components which are important for operation. In one embodiment, the smart devices 100, 200 (see FIGS. 1-2), and in another embodiment, a smart device which is a smart catheter device 300 (see FIG. 3), include (but are not limited to), the following plurality of components:

1. Sensors

In one embodiment, the smart devices 100, 200, 300 (see FIGS. 1, 2 and 3) include a plurality of sensors, such as sensors and/or detectors 106, 206, 306, which include at least one of biosensors, flow sensors, energy receptors/sensors 117, 217, distance sensors 113, attachment sensor 126, etc.

In one embodiment, the distance sensors 113 include at least one of ultrasonic, infrared, laser distance or time of flight light emitting diode (LED) sensors; and the distance sensors 113 derive distance by measuring at least one of a time between signal transmission and receipt by the signal receiver 131 of at least one of an intensity of the signal transmission or a pulse change.

2. Cameras

In one embodiment, the smart medical device includes at least one camera (see cameras 131, 331, for example). In one embodiment, cameras are used so that the user can visually see the position of the smart device within the body.

3. Signal Emitters and/or Receivers

In one embodiment, the smart device 100, 200, 300 includes a signal emitter 101, 201, 301 and a signal receiver 107, 207, 307. In one embodiment, the medical device 100, 200, 300 navigates in the body based on, for example, continuous feedback of transmitted signals from signal emitter 101, 201, 301 to external signal transmitter/receiver 102, or to other medical devices 100, 200, 300 or from transmitted signals from within a target location. In one embodiment, signal emitter 101, 201, 301 emits various types of energy including (but are not limited to) chemical, electrical, radiant, sound, light, magnetic/magneto-inductive, mechanical, thermal, nuclear, motion, and elastic.

4. Steering/Propulsion Systems

In one embodiment, the smart device 100, 200, 300 may include steering mechanism 122, 222, propulsion system 119, 229, 329, including, for example, a propulsion activation mechanism 119, propulsion device 110, and propulsion mechanism/release 135. The propulsion system can be active or passive. In one embodiment, the active propulsion device (i.e., arms 110 in FIG. 1), is activated by a propulsion activation mechanism 119 to propel the medical device 100 to the desired position. In other embodiments, the propulsion system including at least one of chemically powered motors, enzymatically powered motors, external field driven motors, internally mounted miniaturized electrodes, miniaturized electromagnetic pumps, or appendages, etc.

5. Anchoring Device

In one embodiment, the smart device includes at least one anchoring device 129. In one embodiment, the anchoring device 129 is controlled by the program (see below) and/or the user to anchor to a particular position.

6. Energy Storage Devices

In one embodiment, the smart device 100, 200, 300 includes energy storage 111, 211 (internal in FIG. 3 and not shown) via energy receptor 117, 217, 317 which can receive energy externally transmitted to the smart device 100, 200, 300 from external charging sources 112. In one embodiment, a variety of power sources (i.e., object internal energy source 111, 211 or external energy sources 112) can be used to propel the smart medical device 100, 200, 300 —including (but are not limited to) batteries, biofuel cells, thermoelectricity, piezoelectric generators, photovoltaic cells, and ultrasonic transducers. As advances in micro and nanotechnology continue, the range of possibilities for power supply will continue to expand, along with decreasing physical size requirements of the components.

7. Microprocessors, Microcontrollers, Actuator, Accelerometer

In one embodiment, the smart device 100, 200, 300 of the present invention includes a microprocessor 108, 210, 308, memory (i.e., memory 109, 209), and controller (i.e., microcontroller 121 or external controller 130). In one embodiment, signals received or emitted by the smart device 100, 200, 300 can be processed internally using the controller and microprocessor 108, 210, 308 having an internal memory, and a software program which can direct smart device 100, 200, 300 operations. In one embodiment, the program can also be run by external computer system 104, having microprocessor 103 with internal memory 118, connected to a display 105.

In one embodiment, the external signal transmitter/receiver 102 receives signals from the smart device 100, 200, 300 which are processed by controller 130 and inputted to the computer system 104. In one embodiment, the computer system 104 is connected to and controls the external charging source 112. In one embodiment, the computer program (at microprocessors 108, 210, 308 and/or 103) monitors all aspects of the smart device 100, 200, 300 operation, including the transmission/receipt of signals, energy usage, propulsion, steering, sensor operation, etc. In one embodiment, an actuator 133 (not shown in FIGS. 2-3) helps control the navigation and other systems of the smart device 100, 200, 300. In one embodiment, an accelerometer 124 (not shown in FIGS. 2-3) assists in control of the device's 100, 200, 300 acceleration.

8. LIDAR

In one embodiment, the smart device 100, 200, 300 includes a LIDAR mechanism 128, 215, 328. In one embodiment, an integrated lidar scanner 128, 215, 328 senses the physical surroundings and their distances from the smart device 100, 200, 300 by measuring the time requirements for emitted laser pulse to return to a sensor 106, 206, 306. In one embodiment, other inertial sensors 106, 206, 306 record movement with assistance from cameras 131, 331 which provide visual tracking information.

9. Gyroscopes

In one embodiment, the smart device 100, 200, 300 includes a gyroscope 134 (not shown in FIGS. 2-3). In one embodiment a gyroscope measures or maintains orientation and angular velocity of the smart device 100, 200, 300.

10. GPS

In one embodiment, the smart device 100, 200, 300 includes a Global Positioning System (GPS) 123. In one embodiment, a GPS 123, 218, 323 provides the user with positioning, navigation and timing information of the smart device 100, 200, 300.

The specific type and number of these components contained within a given smart medical device are dependent upon a number of factors including (but are not limited to) the specific type of device, device functionality, device form, structure and size, and the desired level of automation (i.e., self-navigation).

11. Tools

In one embodiment, the smart device 200 can include tools which are housed in a plurality of compartments 227 or tool ports 227, and which may include, but are not limited to: a spring 212 actuated cauterization device 213; a delivery device such as injector 229 which delivers an adhesive 204, for example; a clip 205 that is ejected by a lever 221; and pincers 203 that are spring 212 activated. In one embodiment, the tools are controlled by microprocessor 210.

In addition to the various devices and sensors which are integrated into the constructs of the smart device, a variety of ancillary data sources exist, which may play both active and passive roles in smart device 100 navigation. These informational and data sources will be described further below. One relevant example of ancillary data which synergistically contributes to the implementation of self-navigational smart medical devices is the creation of four-dimensional anatomic visualization maps, which was described in the incorporated patents/applications. Since the primary focus of the present invention is the creation of completely automated self-navigating (i.e., autonomous) smart medical devices, the following description focuses on these aspects of the present invention.

Since the principal challenge for any autonomous device is navigating through the diversity of various anatomic structures, it is important that smart medical devices have a method for continuously monitoring and adjusting to the complexity of these various internal environments. Since the human (and animal) body is a complex milieu which is continuously and sometime rapidly changing, smart device navigation must be dynamic in nature and capable of rapid real-time adjustments.

Since the various technologies used for existing smart autonomous devices primarily operate in open-ended environments (i.e., drones), which are not applicable to smart devices which often navigate with margins of error on the order of millimeters, the ability to incorporate real-time anatomic mapping into the smart device 100 navigational strategy is important. While several of the technical components listed in FIGS. 1-3 (which show embodiments of the present invention) play a fundamental role in smart medical device autonomous navigation (e.g., sensors, cameras, actuators, LIDAR), successful in vivo navigation can be dramatically enhanced with supplemental three- or four-dimensional anatomic data.

In one embodiment, as shown in FIG. 3, the smart medical devices may also include medical devices such as a smart catheter 300. The catheter 300 would have similar components on its body to smart device 100, 200 such as a signal emitter 301, camera 331, and LIDAR 328, sensors 306, signal receivers 307, energy receptors/sensors 317, anchoring device 329, a computer system including microprocessor 308 with memory 309, actuator, accelerometer, etc.), a steering mechanism 322, a propulsion activation system 319, distance sensor 313, and GPS 333.

The present invention differs from current practice of positioning medical devices, which includes three primary ways. The first is blind application, where the operator inserts the device without anatomic or visualization cues. An example is the placement of nasogastric tubes, which are routinely inserted in the nasal cavity and advanced into the stomach. A frequently experienced complication is when the tube follows an abnormal course and instead of traversing the esophagus into the stomach, instead enters the trachea (which is the primary airway) and ultimately ends up mispositioned within a pulmonary bronchus. If this goes undiagnosed and not immediately corrected, this can result in tube feedings entering the lung, causing pneumonia. It is for this reason that post-feeding tube placement is typically followed by obtaining a radiograph prior to clinical use.

The second method for conventional medical device placement is through direct intervention (e.g., surgery), in which the operator manually inserts the medical device while directly visualizing its placement. While this almost certainly guarantees correct positioning, it comes at a price and that is the morbidity associated with surgery. An example of this is placement of a medical infusion pump, which will often require days for patient healing and is often associated with some degree of tissue injury and bleeding.

The third method is placement of the medical device under a visualization technique, which typically takes the form of traditional medical imaging technologies such as ultrasound, fluoroscopy, or computed tomography (CT). In this application, the operator follows the path of the smart device in real-time, as it advances, until it reaches its desired destination. At that point in time, the medical imaging is terminated, and the device position is secured. A number of limitations exist with this strategy. Firstly, the positioning of the device may be limited by the technical abilities of the operator or limitations in the visualization technology deployed. For example, fluoroscopy has limitations in the degree of anatomic resolution and granularity. Ultrasound has limitations in both the field of view as well as the depth of anatomy it can visualize. Computed tomography (CT), while providing greater anatomic resolution and depth may be limited by patient body habitus, a variety of artifacts, and the duration of visual guidance. In addition, both fluoroscopy and CT produce ionizing radiation, which can adversely affect both the operator and patient. The operator may be exposed to particularly high doses of radiation for an intervention (e.g., medical device placement), often requiring several minutes. Lastly (and perhaps most importantly), medical devices are often prone to positional change, which can impact their functionality. In current practice, when a device's position changes, it often goes undetected or is ignored. If repositioning is required, it requires the entire manual positioning process to be repeated, which may be time consuming and expose the patient (and medical device) to additional iatrogenic complications.

In contrast to the above methods, in one embodiment of the present invention, smart medical device placement would include a methodology in which the medical device can utilize real-time data and technology to direct its own navigation, with or without external support from an authorized operator. The smart medical device 100, 200, 300 of the present invention accomplishes this by directly visualizing the physical environment in which it travels, can utilize its intrinsic ability to self-correct course as needed, is capable of “hands free” self-propulsion, and is capable of repositioning itself on an as needed basis. These abilities require the smart devices 100, 200, 300 and/or their operators to dynamically process anatomic and positional data on a continuous basis as provided by the present invention. This dynamic data collection, processing, and analysis can be derived from the technology embedded within the smart device 100, 200, 300 (e.g., sensors, microprocessors) and/or real-time data being collected by a variety of external sources which will be further discussed below. The net result is the creation of artificial intelligence (AI) intrinsic to the smart device, which allows it to dynamically self-navigate itself in accordance with its specific task or mission.

A. Defining Navigational and Localization Parameters:

In addition to the embedded sensors and components (i.e., propulsion mechanism, GPS), etc., described in FIGS. 1-3, smart medical devices 100 of the present invention can utilize additional data sources to assist them in their navigation.

In one embodiment, an authorized operator can provide feedback and direct input into the smart medical device navigational controls/complex (i.e., with respect to smart device 100, the steering mechanism 122, propulsion mechanism 119, GPS 123, sensors 106, etc.). This human-derived input is then recorded in data storage/memory 109 and analyzed by the program for the purpose of creating machine learning algorithms, which assist in future in vivo navigation. Since each individual host patient has their own unique anatomic and pathologic variations, the data recorded by the program in data storage 109 can be used for both user-specific and generalized navigational strategies (given the same type of host).

In one embodiment, additional primary data sources for creation of artificial intelligence (AI) navigational algorithms also include (but are not limited to) the specific type of smart medical device, unique device embedded components (i.e., with respect to smart device 100, sensors 106, devices (i.e., GPS 123, anchoring device 229, etc.), cameras 131, etc.), device size and structural components, anatomic region of interest, pathology of interest, device functionality, and types of data being communicated to and from the smart medical device 100 (as well as the specific data sources). Thus, as the data being recorded in storage 109 and analyzed by the program increases in volume, complexity, accuracy, and depth; the degree of sophistication of the resulting artificial intelligence (AI) algorithms similarly increase.

As the lifetime, use, and utility of a given smart medical device continues to expand, so does the data it generates, which provides a valuable method for self-correction and refinement of the AI navigational algorithms. But in addition to this ever-increasing self-generated device navigational data, other smart medical devices 100, 200, 300 can also be valuable data sources as well. In one embodiment, since smart medical devices are fully capable of inter-device communication and data sharing, the anatomic and navigational data being continuously collected by the program (via sensors, etc.) can be derived from an infinite number of external device sources (i.e., system 104, other smart medical devices 100, 200, 300).

In one exemplary embodiment, the smart medical device 300 of primary interest is a vascular catheter 300 (see FIG. 3), which has been inserted into an antecubital vein. The primary purpose of this device 300 is to deliver a dose of chemotherapy to a small one (1) cm liver tumor in the posterior segment of the right hepatic lobe. Since the liver has dual blood supply from both the portal vein and hepatic artery, there are two viable options for targeted navigation to reach the specific location of interest. Based upon previous medical imaging (e.g., MRI), the optimal anatomic target is via the right hepatic artery since a small distal branch is the primary vascular supplier of the tumor in question.

At the time of previous tumor biopsy, a small surgical clip is inserted along the margin of the one (1) cm tumor, which contains signal emitters 301 and receivers 307 (see the incorporated patents/applications). The signals being emitted from this tumor marker provide the equivalent of a beacon, from which the program of the actively navigating smart vascular catheter 300 can synchronize its internal navigation system. At the same time, in this exemplary embodiment, the smart medical device 300 can be instructed to identify the specific hepatic arterial branch supplying the liver tumor, and the program of the smart device 300 may incorporate ancillary anatomic data which has been previously acquired from prior imaging studies (e.g., MRI) while utilizing features such as the camera 331, and making a comparison using the program at the internal microprocessor 308 and/or external microprocessor 103.

In one embodiment, coordination of the external imaging data with the smart device 300 navigational system is carried out by the program. One way is for the program (at the internal microprocessor 308 and/or external microprocessor 303) to fuse the imaging dataset coordinates with the real-time device 300 position in vivo, with continuous feedback provided to the device 300 microcomputer 308 and/or microcomputer 103 as to the device 300 position relative to anatomic location of the pathology in question.

In one embodiment, the program synchronizes real-time data being collected by circulating nanobots (see the incorporated patents/applications), which serve as continuous signal emitters/receivers to produce 4-D anatomic visualization maps (see the incorporated patents/applications). As bidirectional data is transferred between the circulating nanobots and smart medical device 100, for example, using signal transmitters 101 and receivers 107, internal guidance is provided by the program to assist the smart medical device 100 in autonomous navigation to the site of the liver tumor.

In one embodiment, once the smart medical device has successfully navigated itself to the specific location of anatomic/pathologic interest, the next step is for the smart device to position itself in an optimal position for performing its designated function (e.g., chemotherapy infusion). This would represent a unique feature of autonomous medical devices, which requires positional precision which is currently not required or available with existing autonomous technologies.

Using the previous example of targeted chemotherapy infusion within the one (1) cm liver tumor, in one embodiment, both the smart device, and also the specific drug delivery mechanism embedded within the smart device 100 (see the incorporated patents/applications), are to be meticulously positioned to the exact location of the active tumor. In order to differentiate between active and non-active malignant cells, in one embodiment, the smart device could contain a number of miniaturized sensors and devices (see the incorporated patents/applications) capable of performing a variety of individual tasks (see FIG. 2, for example), which collectively perform the functions of navigation, positioning, cellular assay, and drug delivery. Once the smart device has navigated to the tumor location, it would then deploy the cellular collection and assay tools to sample tumor cells in order to identify the specific location of active malignancy.

In one embodiment, once the task has been performed, the navigational system of the smart device would be deployed by the program to specifically position the smart device in direct contiguity with these active tumor cells. But that positioning would then need to be further refined by the program so that the drug infusion device contained within the smart device is directly positioned at the site of active tumor, requiring precision in positioning on the order of 1-2 mm.

In one embodiment, to facilitate this positional alignment, the smart medical device deploys a microscopic marker at the site of active malignancy, which can in turn emit signals to assist in accurate device positioning. In one embodiment, the marker could be deployed from a storage compartment 227 in a smart device 200, for example.

In one embodiment, the present invention provides a plurality of levels of smart device self-navigation, which include:
0: Complete Manual Navigation. Smart device navigation is entirely controlled by operator with no active assistance.
1: Operator Assisted Navigation (A and B). Smart device and associated data provide partial (A) or continuous (B) feedback by the program to operator for navigational assistance.
2: Partial Automation. Limited automation features assist in smart device navigation, which remains primarily under the control of the operator.
3: Coordinated Navigation. Advance automation features allow for smart device to be in part self-navigational, and in part under the control of the operator.
4: Self-Navigation with Passive Operator Assistance. Sophisticated automation features of the program allow the smart device to be self-navigational under optimal conditions, but still require active monitoring on the part of the operator.
5: Complete Autonomous Navigation. Complete autonomy in which the smart device (i.e., the program) is fully self-operational and does not require operator assistance. An emergent intervention option is present for an authorized operator to intervene when deemed necessary.

2. Real-Time Data Analysis

In one embodiment, by embedding a variety of miniaturized devices directly into the architecture of the smart device (see FIGS. 1-3), smart medical devices are capable of self-navigation, in much of the same way that autonomous cars, submarines, or drones, function. Data from the local environment is being continuously collected, recorded, and analyzed by the program, through advanced computer processing external or internal or both, to the device, which in turn provides guidance to the power and steering components of the device.

In one embodiment, the resulting data-driven analysis can also be used by the program to generate three-dimensional (3D) maps of the local environment (i.e., anatomy) encountered during the transit of the device, which can be stored by the program in local or centralized databases for future reference. Since a given host patient will likely experience multiple episodes of smart device deployment over the course of their lifetime, these anatomic maps can be continuously updated and modified by the program, while providing future navigational guidance to other smart medical devices.

In one embodiment, in addition to the data and resulting analyses generated by the program of the smart device itself, external data can provide valuable complementary data to assist in 3-D anatomic guidance and smart device self-navigation. A variety of anatomic mapping and visualization tools can be used for this purpose, including currently existing medical imaging technologies (e.g., CT, ultrasound, MRI) as well as futuristic visualization tools, such as the 4-D visualization maps described in the incorporated patents/applications.

In one embodiment, there are a number of iterations of such anatomic visualization technology which can be incorporated into the smart device of the present invention. In the simplest iteration, the smart medical device navigates on its own and incorporates readily available anatomic data (e.g., MRI imaging dataset) using the program, into its own internal microprocessor for the purpose of the program analyzing and anticipating future directional change and modification along its intended course. In essence, this is a one-way flow of information from the external anatomic data source to the smart device.

In one embodiment, in a modification of this iteration, a third party (i.e., authorized human operator) can serve as an intermediary between the external anatomic data source and the smart medical device. This operator would have access to both the smart medical device derived real-time data (e.g., video, sensor, LIDAR) along with pre-existing anatomic data to provide external guidance to the program of the smart medical device.

In one exemplary embodiment, a smart vascular catheter 300 is tasked with local drug delivery to a diverticular abscess within the wall of the sigmoid colon, and knowing the intended task and anatomic location, the operator can serve as a guide to assist the smart device 300 in its navigation. If, for example, the smart device 300 was to miss the turn off in the arterial branch supplying the portion of the sigmoid colon containing the abscess, the operator may provide guidance and recommended course correction to the smart device microprocessor 308, by sending the appropriate instructions via signals from signal emitter 102 to the smart device 300 to reroute its course.

In one embodiment, the unilateral data flow previously described from the external anatomic dataset (+/−operator) (i.e., microprocessor 103) to the smart device 300 is replaced by bidirectional flow between the pre-existing anatomic dataset (i.e., storage 118) and the smart device 300. In this instance, microprocessors 308, 103 within both systems are analyzing data and interactively communicating with one another to optimize smart device 300 navigation.

In one embodiment, the program of the smart medical device is sharing its continuously derived internal data and intended navigational course with the computer 103 tied to the anatomic dataset, which program can now analyze and visualize on the display 105 in real-time the exact location and course of the smart medical device 300, while providing real-time feedback. Once again, a modified version can supplement this bidirectional interaction with that of an authorized operator, who has simultaneous access to real-time data being collected by the smart medical device 300, along with the pre-existing external anatomic visualization data stored in database 118.

In one embodiment, since smart medical devices can contain embedded signal emitters and/or receivers, highly valuable 4-D locational data is continuously being transmitted and received by the program. This provides a continuous update of the smart device's in vivo positioning within the host subject for program navigational monitoring and adjustment.

In one embodiment, during the course of the smart medical device navigation, the program can generate from the real-time sensor-derived data, its own anatomic visualization map, which can be cross correlated by the program with external visualization maps (e.g., CT imaging dataset) for variations and/or discrepancies and shown on the display 105. As the smart medical device navigates through the host subject, the anatomic visualization data can in turn be used by the program to edit the external anatomic dataset (i.e., at data storage 118); in effect, providing a new and enhanced version of this dataset. Since conventional imaging datasets are routinely static in nature, they do not always reflect the most current and up to date state of the anatomy and/or pathology, particularly in the setting of a rapidly changing medical condition. This embodiment provides an example where the bidirectional communication between the smart medical device and external dataset can be mutually beneficial and provide enhancements to the static imaging dataset which is not available in current practice.

In one embodiment, the use of external anatomic datasets to assist smart device navigation need not be limited to a single data source, but instead can involve multiple ones. One could in practice have the program combine or fuse multiple disparate datasets (e.g., CT, ultrasound, nuclear medicine, MRI) into a single all-inclusive anatomic visualization map for smart medical device navigational assistance. Using existing rigid and nonrigid techniques, multidimensional anatomic visualization maps can be created by the program.

In one embodiment, an even more advanced iteration of the present invention can be created, where continuous interaction and data sharing is being performed from multiple sources which provides the smart medical device with far more advanced self-navigational capabilities. In the incorporated patents/applications, a novel methodology was described in which large numbers of circulating nanobots (or microbots) were introduced into the host subject for the purpose of creating a real-time and dynamic 4-D visualization map. If one was to have the program combine the data and intrinsic intelligence of smart medical devices with such a nanobot derived 4-D visualization maps, a far more sophisticated autonomous smart medical device navigational system can be created. The details of this embodiment of the present invention are provided hereinbelow.

In one embodiment, as the nanobots/smart devices circulate throughout the host subject en masse, thousands (or even millions) of signals are continuously being emitted, received, and processed to the smart devices. This in turn allows the program to generate a 4-D visualization map which is being continuously upgraded to reflect real-time anatomy and pathology, along with the physiologic (and non-physiologic) changes which are constantly occurring.

In one embodiment, with the introduction of a single or multiple smart devices, an entirely new set of signals are being emitted, received, and processed by the program, which are intrinsic to the smart device(s) and its (or their) changing position(s). The continuously changing position of the smart device(s) and their relationship to surrounding anatomy and pathology can now be tracked and analyzed by the program in great detail and is not available in conventional methods.

In one embodiment, continuous feedback can be provided to the navigational system of the smart medical device(s) by the program, knowing the device's intended course and function. Thus, large numbers of data are continuously recorded and analyzed, for the purpose of providing real-time anatomic analysis and potential impediments to navigation, along with recommendations for course correction.

In one embodiment, in addition to the 4-D visualization map created by the program controlling the circulating nanobots, a comparable visualization map is created by program. By the program combining these two maps, subtle differences can be reconciled and used to edit and refine the individual maps. Throughout the navigational course of the smart medical device, data is being recorded and stored in a database by the program, and analyzed by the program, for future use. This may be valuable for both this individual smart medical device in its future missions, as well as other smart medical devices. Since the intended location, functionality, and structure of smart medical devices will vary, the navigational data derived from an individual smart medical device's actions by the program, can be applied and modified by the program, in accordance with each unique device's functionality, location, and mission.

In an exemplary embodiment, the navigational and anatomic data derived by the program from the smart vascular catheter's mission to deliver antibiotics to the diverticular abscess, is now being applied to a new purpose, that in which a separate smart medical device is being used to travel to the same location (i.e., sigmoid colon), but for a different purpose, namely repair of a small perforation is the sigmoid colon wall. In order to perform this job, the smart device being deployed is of a larger size and dimension, allowing for the embedded surgical device required for the repair.

In this exemplary embodiment, knowing the anatomic location and path of intended navigation, the program can determine that the larger smart device size will require an alternative travel plan, and this can be plotted by the program prior to the deployment, based upon detailed knowledge of anatomy and the pathology in question. In some respects, this can be likened to a roadmap one can create when planning a trip from point A to point B, only in this case, the requisite data is on a far more granular level. Without pre-existing data derived from the 4-D visualization map, proactive planning would be limited.

In this exemplary embodiment, at the same time, detailed knowledge of the anatomy, pathology, and required smart device functionality will also prove important in proper selection of the smart medical device being deployed. If, in this example, the size and dimensions of the smart surgical device exceeds existing physical parameters, an alternative smart device must be selected, which can accommodate to the real-world limitations that exist.

In the exemplary embodiment, once in route, the program of the smart medical device will utilize the combination of internal and external data available to self-navigate to its intended location. Continuous real-time data updates and analyses by the program will allow the smart device to self-correct and adjust course as needed.

In this exemplary embodiment, the result is that smart medical device navigation of the present invention can utilize a variety of data sources whose origins include (but are not limited to) real-time data collected by the individual device in question, pre-existing traditional anatomic data sources (e.g., CT, ultrasound, MRI), pre-existing smart device anatomic data sources, real-time data actively being collected from other circulating smart devices (including nanobots and microbots), and data from stationary smart devices (e.g., prosthesis, pacemaker, surgical clips). These various data sources can be stored and analyzed by the program in local and centralized databases and be readily accessible to authorized smart medical devices in transit, so as to allow them to refine and update their navigational course, in association with the anatomic location and context of their intended mission. At the same time, newly acquired real-time data being collected by circulating smart medical devices can also serve to have the program iteratively refine and update pre-existing anatomic data sources to more accurately state current anatomy and pathology.

C. Artificial Intelligence

Some of the key components of the present invention include (but are not limited to) its composition and structure, technical components, cognition, and adaptability. Collectively, these features create the unique capability of creating intelligent, adaptable, and autonomous smart devices, which go far beyond existing technology.

In one embodiment, cognition is a core component, for it allows smart medical devices to carry on functions and activities which can be completely (or partly) independent of human operator assistance. In one embodiment, a variety of current and future artificial intelligence (AI) techniques can be used to allow smart devices to do so, which will obviously expand in scope and complexity as the field of AI continues to advance. In one embodiment, smart medical devices of the present invention can utilize AI in a variety of ways including (but are not limited to) autonomous navigation, medical diagnosis, and treatment.

Generally, artificial intelligence (AI) refers to computer software which can think and act independently, in some ways replicating the actions of the human brain. But in more practical terms, AI (in its current form) refers to computer software which relies on algorithms to analyze data, identify patterns, and make predictions. This process of data-driven learning is often referred to as machine learning, which provides adaptive knowledge by the computer in accordance with changing data patterns and analysis.

In one embodiment, one important and relevant form of AI to the present invention is convoluted neural networks (CNN), which is a form of deep learning algorithms which analyze input imagery in a manner analogous to the visual cortex of the human brain. While CNN has been used in a variety of applications, it is particularly relevant for use in both the navigation and diagnostic components of the present invention.

In one embodiment, a number of other forms of AI are potentially applicable for use in the present invention including (but are not limited to) segmentation analysis, radiomics, principal component analysis, support vector machines, deep learning, computer vision, Bayes decision rule, K nearest neighbor, and sensor fusion.

a. Exemplary Embodiment or Use Case:

In one exemplary embodiment, which illustrates how these various forms of AI can be incorporated into autonomous smart medical devices of the present invention, a smart vascular catheter 300 which has been introduced in the setting of an acute stroke, resulting from occlusion of the right middle cerebral artery, is used. The goal is to autonomously advance the catheter to the origin of the right middle cerebral artery, where it will locally administer a thrombolytic agent for dissolving the occlusive thrombus responsible for the acute stroke.

In one embodiment, the smart catheter 300 is inserted into the right femoral vein which provides a readily accessible entry site. Once inserted, the catheter 300 must follow a pre-arranged or pre-programmed course, as instructed by the program, in order to reach its ultimate destination. The amount of pre-existing data related to the host patient's anatomy, physiology, and pathology can be highly variable. As a result, the smart catheter 300 should be capable of self-navigation independent of ancillary data, which is an important feature of the present invention. While ancillary anatomic data sources can serve fundamental roles in defining anatomy and assisting in smart device navigation, an autonomous smart medical device should be fully capable of self-navigation, with or without the support of ancillary data.

In this exemplary embodiment, the host patient does have available data in the form of a recently performed head CT exam, which provides both brain anatomic and pathologic data accessible to the program. The remaining host patient anatomy and pathology outside of the brain is not known at the time, requiring the smart catheter 300 to self-navigate based on its own internal capabilities.

In the exemplary embodiment, as shown in FIG. 3, a number of embedded technologies are available to assist the smart device 300 in its autonomous navigation, which can be synergistically assisted by a variety of AI programs. One such relevant program allows access to a database 309, 118 of a compendium of anatomic atlases which define conventional anatomy, as well as congenital anatomic variations. Since congenital variations in arterial anatomy are relatively common and can frequently affect smart device navigation, it is important that such reference data be available in real-time to the program, for the program to perform analysis and make necessary navigation course adjustments without incurring iatrogenic vascular injury or unexpected time delays.

In the exemplary embodiment, as the program of the smart device is actively obtaining real-time data relative to its immediate environment, the data is correlated by the program with anatomic reference data to identify any unexpected anatomic variation, requiring course correction. At the same time, the data being actively collected by the program will also identify pathologic states, which may require immediate intervention or modification in navigational strategy by the program. A few examples of anatomic variation relevant to this particular example may include (but are not limited to) a right sided aortic arch, unusually high origin of the right common carotid artery, or duplicated middle cerebral artery. Regardless of the anatomic variation encountered, the ability of program of the smart device to correlate real-time navigational data with anatomic reference data provides the capability of rapid, reliable, and safe course correction.

In one embodiment, in many circumstances, anatomic variations occur in multitude and sometimes in a predictable pattern. By having the ability of the program to consult anatomic reference data, concomitant anatomic variations may be anticipated by the program, a priori. As more and more data are collected, the derived knowledge collected by the program, provides smart devices with the ability to improve navigational performance and make adaptive change in a rapid and intuitive fashion.

In one embodiment, if prior medical imaging data is available for a given host patient, this data can be directly incorporated by the program into the individual patient anatomic visualization map and used for proactive navigational planning. In the event that historical imaging data is found to be deficient in some form, the newly acquired smart device data is used by the program to update and revise the historical imaging data, so as to provide an up-to-date visualization map which accounts for both anatomic and pathologic change.

In one embodiment, another important and novel application of the present invention is the ability of the autonomous smart medical device to adapt to its local environment and modify its function accordingly. To illustrate possible scenarios for how this real-time adaptive feature may work, in one exemplary embodiment, consider that the smart catheter 300 is tasked or instructed by the program with local infusion of a thrombolytic agent at the site of middle cerebral artery occlusion. In this example, the smart device 300 has now autonomously navigated its way from the femoral vein to the heart, thoracic aorta, and common carotid artery. As the smart device 300 travels from the common carotid artery to the origin of the right internal carotid artery, it detects using its sensors 301, camera 331, or other features, that unexpected pathology in the form of high-grade soft plaque in the right carotid bulb and proximal internal carotid artery. This plaque is important for two reasons. First, it serves as an impediment to navigation and secondly, the soft nature of the plaque serves as a high risk of detachment and embolization, which in itself increases the risk of acute stroke.

In this exemplary embodiment, as the smart catheter 300 attempts to pass through the point of obstruction caused by this high-grade soft plaque, the risk of iatrogenic complication (i.e., embolization of a broken off plaque fragment) may be too great and result in aborting the planned mission unless a safer alternative strategy can be identified and implemented.

In the present invention, since smart devices can have a variety of embedded miniaturized devices and functionality, the opportunity for diagnosis and intervention is quite extensive. In this particular exemplary embodiment, one solution would be to utilize a smart device with the capability of more accurately visualizing, quantifying, and analyzing the carotid artery plaque in question.

In the exemplary embodiment, if the already deployed smart device 300 does not have the required capability to do so, a second smart device 100 which does have these capabilities can be deployed to site of anatomic/pathologic interest. Examples of such embedded miniaturized devices may include, but are not limited to, video cameras 131, ultrasound transducers (not shown), volumetric measuring devices (not shown), and biosensors 106.

In the exemplary embodiment, the derived data can be used by the program to determine the optimal course of navigation as well as any intervention options which may prove beneficial. In this particular example, the program-calculated degree of luminal stenosis caused by the plaque is 80% (i.e., high grade), which effectively reduces the navigable luminal diameter to only 12 mm. With the smart device possessing a diameter of 10 mm, this means that even the slightest deviation off course, will cause the smart device 300 to brush against the plaque and potentially cause disruption and embolism. Based on the real-time data assessment by the program, the options available to the smart device 300 include finding an alternate route to reach its intended destination or aborting the mission and try to find an alternative therapeutic course of action.

In conventional interventional vascular practice, guidewires are inserted into the lumina of the catheters which can be manually manipulated and advanced by a skilled interventional radiologist or cardiologist, thereby providing the catheter with added torque and/or flexibility. However, in the present invention, the ability of the program of the smart device to actively analyze local environmental conditions and proactively adapt would provide an entirely new and novel solution.

b. Increase the functional luminal diameter by reducing the point of obstruction.

In one embodiment, an alternative strategy to the above difficulty is to take action directly onto the underlying pathology causing the obstruction. In this exemplary embodiment, the smart device would employ its therapeutic functionality which is aimed at reducing and/or eliminating the offending atherosclerotic plaque and by extension, increasing the native vessel luminal diameter, allowing it to freely navigate the point of obstruction.

In one embodiment, to accomplish this task would be to deploy a cutting tool (i.e., cauterization tool 213) or drill (not shown) embedded in the smart device 200 to remove the offending plaque. Since the plaque being removed could serve as a potential source of downstream embolus, this strategy must incorporate a method to trap and/or collect plaque fragments to avoid such a complication. This could be done through deployment of a fine mesh or net 219 (i.e., deployed from an internal compartment 227 using a spring 212, for example) which traps the detached plaque or alternatively deploy a vacuum apparatus 220 (i.e., deployed from an internal compartment 227 using a lever 221) which collects and stores plaque fragments for later disposal.

In one embodiment, the smart device in question may not possess the functional and/or technical capabilities of reducing the plaque burden and/or safely collecting plaque debris. However, this does not eliminate the possibility of performing this task, which may be determined to be the best option for smart device navigation across the point of obstruction. In this scenario, recruiting an additional smart device(s) may be required, which could act in tandem with the original smart device to complete the task at hand.

The above exemplary embodiments illustrate an important and novel application of the present invention, which is the ability to perform multi-directional communication and coordination of smart device activities. In one embodiment, the smart medical device can possess the ability to communicate (i.e., through wireless transmission) with a central computer (i.e., computer 104), an authorized human end-user, and/or other smart medical devices. This multi-directional communication serves a number of purposes, one of which is the coordination of multiple smart device interactions.

In this exemplary embodiment, the smart device of interest (i.e., primary smart device) may communicate a number of data including (but are not limited to) the finding of navigational obstruction, the specific anatomic location of the obstruction, details related to local anatomy (e.g., anatomic variation, vessel size, vascular tributaries, etc.), and details related to the specific obstruction (e.g., type of pathology, dimensions, composition, etc.). This data can in turn be analyzed by the program to determine the intervention options as well as required supporting technologies.

In one embodiment, the resulting analysis by the program identifies an additional smart device(s) (i.e., secondary device/s) which may be able to assist the primary smart device in plaque reduction/removal. This may include a single device with requisite drilling and vacuum capabilities or multiple devices to provide the bevy of actions required (e.g., one device for plaque removal and another device for plaque collection and storage).

In one embodiment, anatomic data supplied by the primary smart device can then be supplied to the secondary device(s) by the program in order to facilitate their own autonomous navigation to the anatomic/pathologic location of interest. As the secondary device(s)′ approach to this desired location, the primary smart device may assist their navigation through the transmission of signals which are received by the secondary device's and guide their navigation —analogous to a beacon.

In one embodiment, upon arrival, the primary and secondary smart devices communicate between one another to coordinate the desired activity. In the setting where two secondary devices are being deployed, the device responsible for plaque removal (i.e., the driller), positions itself alongside the targeted plaque while the device responsible for plaque collection and storage (i.e., the retriever) positions itself so that dislodged plaque is retrieved by its collection device and transferred to a storage reservoir (i.e., compartment 227) for eventual elimination.

In one embodiment, the primary device simultaneously collects anatomic visualization data which is processed by the program to determine the change in vessel luminal diameter pre- and post-intervention. Once the program has determined that the intervention has been successful and the native vessel is now safe for navigation, the secondary devices are notified of task completion. In select circumstances, it may be determined by the program that placement of an intraluminal stent at the site of plaque formation and obstruction may be required to prevent future plaque reaccumulating and obstruction. In this situation, another smart device may be utilized to deliver and deploy the stent at the designated location.

In one embodiment, once the desired intervention has been completed and data obtained for the program to validate successful task completion, the primary smart device is now free to resume navigation through the location of prior obstruction, while the secondary devices are free to return to their designated site of origin.

In one embodiment, in the same manner, these same communications and actions can be performed under the supervision and/or guidance of an authorized human operator. As previously stated, a spectrum exists regarding the degree of autonomy of smart medical devices, which can be applied to a variety of functional applications, one of which is navigation. In the aforementioned use case, the smart devices acting in coordinated concert with one another were completely autonomous and devoid of human supervision and/or direction. In a less autonomous state, the communication, navigation, and coordination of activities can be supervised and/or directed by an authorized human operator. In such a scenario, the data and communications being shared by the program could involve the human operator as an intermediary. Irrespective of whether these smart devices are completely or semi-autonomous, the application of the present invention remains the same—i.e., primary and secondary smart devices can communicate and interact with one another in the coordination of their navigation, data collection, and performance of duties.

Thus, in one embodiment, the smart devices of the present invention utilize their vast array of cognitive, technical, therapeutic, and adaptive tools to proactively analyze a given challenge and intervene. Once the operation has been successfully completed, the primary smart device continues to navigate to its intended destination (which in this case is the right middle cerebral artery), where it carries out its designated task (which in this case is local infusion of a thrombolytic agent at the point of arterial obstruction).

c. Detaching subcomponents of the device to reduce overall size.

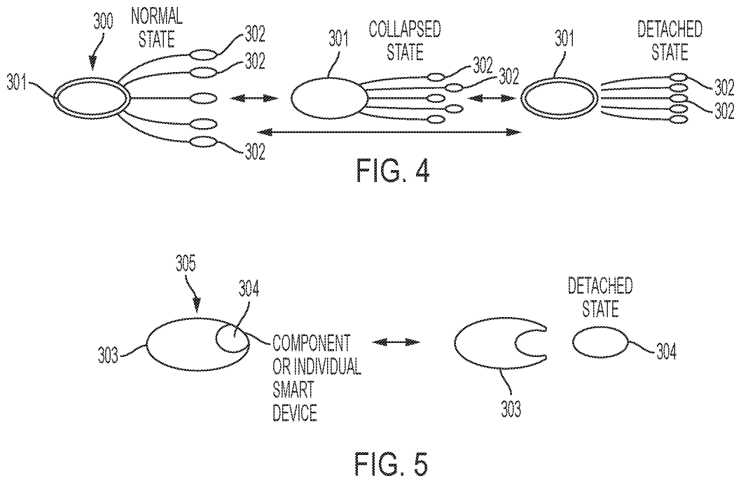

In one embodiment of the present invention, a given smart medical device can reduce its overall size and/or footprint by splitting up into individual subcomponents, in order to navigate through a region of smaller dimensions. In one embodiment, as shown in FIG. 4, a smart device 300, with a main body 301 and a plurality of subcomponents 302, attached to the main body by tethers, can narrow its overall profile by navigating into close proximity, and then if desired, the subcomponents 302 can detach from the body 301 for individual navigation.

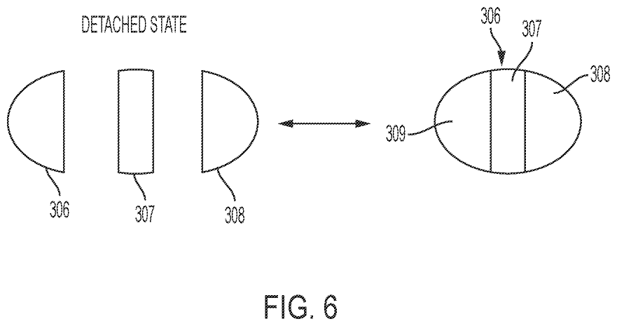

In another embodiment, as shown in FIG. 5, a component or individual smart device 304 attached to a body 303, can detach so that the smart device 305 can be navigated separately.

In these exemplary embodiments, if these detached subcomponents 302, 304 each possess their own navigational components, they can independently traverse the restricted anatomic region and if required, re-attach (or re-assemble), with the primary smart device component at a site downstream from the area of restriction.

In one embodiment, to accomplish this functionality, the various device subcomponents are constructed in an articulated manner which allows for them to become physically detached and reattached as necessary. If markers are positioned at the edges of these subcomponents 302, 304, this would ensure that the re-attachment process is orderly and accurate. Once the re-attachment has been successfully completed, a reversible locking mechanism (not shown) can be deployed for the purpose of strengthening the connection between individual subcomponents.

Although the example of a simple vascular catheter with a linear configuration, would show minimal benefit, when a smart device has a more complex configuration (e.g., pacemaker, bifurcated vascular stent), the benefits of detachment and reattachment becomes more pronounced.

d. Aggregating multiple smaller devices into a single large fully functional device.

In one embodiment, smart devices of the present invention come in a variety of different sizes, from microscopic nanobots to large conventional macroscopic smart devices. When smaller smart devices such as microbots or nanobots are being used, having the ability to merge or coalesce multiple individual smart devices and/or subcomponents into an aggregate smart device creates the ability to create increased functionality and strength which may not be available with individual smaller smart devices acting independently. This aggregation capability is described in a preliminary way, in the incorporated patents/applications.

In one embodiment, under some circumstances, multiple individual smart devices and/or subcomponents can be aggregated at specific anatomic location of clinical concern. If, for example, smart devices are being used to deliver chemotherapy within an intraventricular brain tumor, it may be difficult to deliver in vivo a large smart device with combined infusion and storage capabilities given the physical constraints of the blood-brain barrier. One way to circumvent this challenge (short of invasive brain surgery), is to aggregate multiple smart microbots and/or nanobots at the tumor site for local drug delivery.

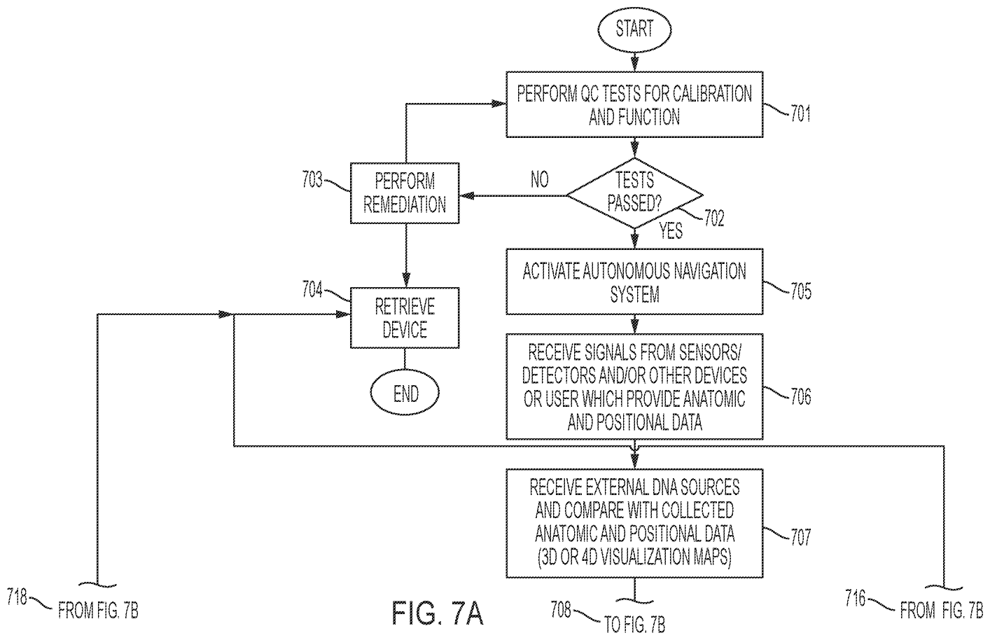

In one exemplary embodiment, as shown in FIG. 6, three (3) smart devices 306, 307, 308 may be aggregated into a single smart device 308 and kept together with a locking mechanism.

In another exemplary embodiment, if a large smart device (i.e., similar to smart device 309) includes a variety of subcomponents which when connected create navigational size limitations, such as a cardiac pacemaker, or other smart device with delivery systems etc., if each of the various subcomponents possess its own navigational capability, they can be directed to the anatomic site of interest and joined in accordance with the given device roadmap, in a manner analogous to joining individual pieces of a jigsaw puzzle. One the construction process has been completed, quality control testing can be done by the program to ensure both the configuration and functionality of the device are intact and accurate.

e. Expanding the device at the desired anatomic location.

In one embodiment, after collapsing a smart device by detachment of various components at a specific area of size limitation, the smart device in transit may exist in a non-functional contracted state which requires full expansion before it can be fully operational. An example of such a device may include an inferior vena cava (IVC) filter (which is used to trap emboli from the lower extremity veins) or an endoluminal bifurcated arterial stent (used to establish patency and/or treat an abdominal aortic aneurysm). In both examples, the footprint and overall size of these fully functional smart devices may limit their navigation in vivo. If, however, in one embodiment, one was to introduce the smart device with autonomous navigation capabilities in a collapsed state, it could freely navigate the required blood vessels and when properly positioned at its destination location, it could be fully expanded (e.g., deploying internal components through spring loading or hydraulic mechanisms, or other mechanisms) for full functionality and attachment mechanisms deployed for positional stability.

However, in lieu of these various options to circumvent anatomic size restrictions, in one embodiment, an alternative option is to change the desired navigation route. In this exemplary embodiment, consider a situation where the plaque in the internal carotid artery is so severe, it presents a complete and impenetrable option for antegrade passage.

In one embodiment, an alternative option is for the program to identify an alternative navigation route in order to reach its intended destination. This embodiment requires the smart device program run by the microprocessor to analyze host anatomy, identify potential alternate routes, correlate associated pathology along these alternate routes (if anatomic/pathologic data is readily available), and coordinate any requisite intervention.

In this exemplary embodiment, the smart device encounters high-grade stenosis within the proximal right internal carotid artery (as determined by sensors/camera, and the program analysis of the data) and the program determines the best course of action is to identify and alternative navigation route. Even if the individual host patient anatomic data is not available for analysis, the program of the smart device can data mine available anatomic atlases and the program can plot a theoretical alternative course and make any required course corrections along the way, as it continuously gathers real-time data specific to host anatomy.

In one embodiment, based upon this anatomic reference data, the program of the smart device identifies an alternative navigation route, in which it reverses direction, navigates its way back to the aortic arch, and enters the right subclavian artery, followed by the right vertebral artery. In this exemplary embodiment, it follows the course of the right vertebral artery to the basilar artery, which leads to the circle of Willis (in the brain), where it enters the right posterior communicating artery, which finally merges with the right middle cerebral artery. Once it arrives at the origin of the right middle cerebral artery, it performs its intended/instructed task of infusing the thrombolytic agent for dissolution of the occluding embolus which has caused the acute stroke.

In another exemplary embodiment, consider that the smart device encounters a congenital anatomic variation such as fetal origin of the right posterior cerebral artery. Upon the program analyzing the data and recognizing this variation (from its own internal analysis along with supplemental data supplied by an anatomic reference atlas), a new course adjustment is made by the program, which redirects the smart device from the circle of Willis to the contralateral left posterior cerebral artery, left anterior communicating artery, right anterior communicating artery, and to its final destination, which is the origin of the right middle cerebral artery. While the end result is the same, the anatomic pathway required to get there changed in accordance with host anatomy, requiring real-time anatomic data collection, analysis, and course correction by the program.

f. Device Removal/Extraction

In the previous exemplary embodiment, smart medical devices were actively deployed for performance of a specific task. Once the task/s was completed, the smart device would likely be recalled for removal from the host. This entails navigation to a predefined anatomic location for excretion or extraction (see the incorporated patents/applications). A number of anatomic and physiologic pathways exist for normal biologic excretion including (but are not limited to) the gastrointestinal tract, urinary system, respiratory system, and skin.

In one embodiment, the location of smart device removal from the host is in part predicated by the physical size and structure of the smart device, which can be highly variable. At one extreme are conventional medical devices (e.g., pacemaker, catheter, mechanical pump) whose size limits the extraction methods. At the other extreme of smart devices are nanobots. as described in the incorporated patents/applications, which are only about 0.1-10 micrometers in size, for example, which is the size equivalent of a single cell. In between these two extremes are microbots, which in one example, are less than one (1) millimeter in size, which allows unimpeded travel throughout the human body. As a result of their small sizes, smart nanobots and microbots (which can collectively be thought of as miniaturized smart devices) can be eliminated or extracted from the host through the gastrointestinal, urinary, or respiratory systems, as well as the skin.

In one embodiment, the effective lifetime of smart devices is in large part determined by their power sources, which can be internal or external in nature. Examples of external power supply 112 were described in the incorporated patents/applications, in which external power supplies 112 can be used to effectively recharge circulating smart devices as they navigate in proximity to the external power supply 112. In the event that a smart device was either powerless or defective, it would either require intervention for return of power or face elimination/extraction. The methods and options for smart device elimination or extraction are discussed in greater detail below.

D. Tracking Technology.

In one embodiment, the present invention has the novel capability tied to autonomous device navigation, of tracking and continuously monitoring smart medical device location as it navigates in vivo within the hoist patient. Sensors embedded within the smart device can emit signals which can be retrieved by internal and/or external receivers for device localization in real-time. While other autonomous technologies such as drones, submarines, and vehicles can be localized with GPS technology, prior art in vivo devices are not trackable with existing GPS technology, thereby requiring an alternative technology for continuous real-time tracking.

If conventional medical imaging technologies (e.g., CT, MRI, X-ray) are used to localize medical device positioning, they are limited by their static nature, which precludes continuous device localization over a prolonged time period. For conventional in vivo devices which are actively moving, these static visualization technologies are impractical and too limited. An alternative technology is therefore required which can actively and continuously reassess and pinpoint location of devices such as the actively mobile smart devices of the present invention, while also possessing the ability to communicate with the smart device in transit.

In one embodiment, as the smart devices navigate throughout the host anatomy, their specific location can be tracked and monitored by the program through the signals they emit, which in turn can be mapped by the program on 3-D and 4-D anatomic visualization maps. In the incorporated patents/applications, a methodology was described for creation of a 4-dimensional visualization map using circulating smart nanobots, which can be readily applied to the present invention. This technology would create a method for continuous real-time smart medical device localization, which in many ways would be analogous to the GPS tracking of autonomous vehicles. One major improvement of the present invention's visualization and location tracking would be the absence of blind spots which are commonly encountered with GPS (e.g., as an autonomous car drives through a tunnel).

In addition to use of external anatomic visualization data for self-navigation, in one embodiment, the program of the smart devices of the present invention can also utilize their internal device-acquired anatomic data and a variety of artificial intelligence techniques, to actively track, monitor, and analyze smart device location in real-time as well as analyzing performance of its various functions. This ability of the program to surveille smart device movement and activity provides an important and important component of the invention, which to a large extent does not exist with other autonomous navigation technologies.

In one embodiment, some of the primary applications in which this tracking function can be used include (but are not limited to) smart device quality assurance, supervision, feedback, and retrieval. This supervisory and consultation feature of the tracking tool can be performed autonomously and/or with human interaction.

In one embodiment, the quality assurance component of the program is designed to monitor smart device function and performance, in order to assure that the smart device is properly performing its assigned duties. As is the case for any technology (or human), errors may occur. The sooner they are recognized by the program and remedied, the better the effect on clinical outcomes. Some common errors that can take place which the program is capable of ameliorating or curing, with the appropriate quality assurance steps, are faulty navigation, device-induced iatrogenic complications, mechanical breakage, loss of power, and faulty/non-functioning components, as discussed below.

In one embodiment, the tracking tool and its bidirectional communication capabilities of the smart device of the present invention provides a way with which the smart device location and performance can be continuously assessed by the program. An important point is that both the device in its entirety as well as its sub-components can be continuously analyzed by the program for deficiency. This is because the construction of smart medical devices allows for active monitoring of individual components and segments of the device.

In one exemplary embodiment, an inferior vena cava filter is being monitored by the smart device, and in the event that one of its struts was to break off from its base, then sensors contained within this broken strut can be continuously monitored by the program. If the broken strut was to detach itself from the original device based on program instruction, and travel upstream (along the normal flow direction of venous flow), the tracking tool of the smart device provides the ability to continuously monitor locational change. At the same time, the program can monitor via the visualization map, any potential complication caused through this unintended migration, such as vascular injury and/or bleeding.

In this exemplary embodiment, given the potential for iatrogenic injury by this migrating broken strut, it is important that it be retrieved as soon as possible. In current practice, a broken strut frequently goes undetected and is often difficult to localize. However, in the present invention, the tracking function of the program of the smart device and its subcomponents provides the ability to promptly detect mechanical breakdown, continuously monitor location, identify directional movement and velocity, quantify potential for iatrogenic injury, and devise an interventional strategy based upon current and future anatomic locations, device size and structure, and injury potential.

In this exemplary embodiment, based upon knowledge of the size, morphology, and location of the broken strut, an intervention strategy can be implemented by the user and/or program. Using the tracker's ability to continuously monitor positioning, a separate smart medical device capable of retrieval can be dispatched by the user and/or program. With continuous communication between this retrieval device and the broken strut tracking mechanism, the retrieval device can autonomously navigate to the specific location of the broken strut. If signal emitters are contained within the broken strut, this localization process can be further enhanced by the program tracking the signals being emitted.

In this exemplary embodiment, by strategically positioning signal emitters and/or receivers in a variety of positions throughout the footprint of the smart device and creating articulated segments in the smart device construct, one can effectively create the equivalent of multiple individual smaller smart devices within a larger all-inclusive smart device (see FIG. 6). This would create the ability for individual device sub-components to become detached (both intentionally and unintentionally) from the native smart device, yet still function autonomously. An analogy can be made to a rocket which intentionally loses its boosters after takeoff, except in this case the detached segment of the smart device can continue to function independently, in accordance with its intrinsic functionality and embedded miniaturized subcomponents.

In one embodiment, this ability to effectively shed off functional subcomponents which may possess autonomous capabilities is another unique feature of the present invention and may have a number of clinical applications. Once the parent smart device has been inserted into the host patient through a large entry portal, detachment of individual subcomponents can take place through external direction (via an authorized operator inputting commands) or internally (via the program of the smart device).

In one exemplary embodiment, to illustrate how this functionality might work, take a cardiac atrioventricular pacemaker which is inserted via the patient's right internal jugular vein with the plan for autonomous navigation to its intended destination within the right atrium and ventricle. The pacemaker is inserted in a contracted or collapsed state, in order to reduce its footprint and allow passage within the narrowly confined space of the superior vena cava. Under optimal conditions, the collapsed device would autonomously navigate itself into the heart and once it reaches the right atrium, it releases (i.e., deploys) the atrioventricular lead (which could take place either via a spring or hydraulic internal mechanism). The device then proceeds into the nearby right ventricle, where the ventricular lead is released (i.e., deployed). Once both leads have been released, the device self-navigates itself into its final position and becomes activated and fully functional.

However, in this exemplary embodiment, suppose a problem takes place with the self-navigation process, so that the released right ventricular lead becomes mispositioned, or even breaks off from the native device, rendering it ineffective. Malpositioning can potentially be resolved by reorientation, whereas breakage may require retrieval and/or replacement in order for the pacemaker to function properly. In the event that the atrial pacemaker lead has the ability to autonomously navigate from the core device, it could navigate on its own to the correct anatomic positioning and then reattach itself to the core device. Alternatively, as before, a retrieval device can be sent to collect the broken lead, while deploying a new lead with autonomous capability, which navigates to the desired position and attaches itself to the core pacemaker device.

In an alternative exemplary embodiment, the core pacemaker device in its collapsed state may encounter an unexpected obstruction to passage, which prevents the full deployment and release of the individual leads. As an example, suppose a venous web or scarring is encountered which effectively reduces the navigable lumen of the superior vena cava by 50%. In order to navigate through this point of obstruction, the smart device (even in its collapsed state) would have to reduce its overall diameter by 30%. One strategy to do so would be for the pacemaker to effectively break apart (i.e., deconstruct) into its subcomponents (see FIG. 6, for example), each of which could autonomously navigate past the point of obstruction and then reconstruct into the native pacemaker structure after traversing the point of obstruction.

In one embodiment, if each of these subcomponents possesses the necessary miniaturized devices for self-navigation and communication as in the present invention, they could detach themselves from the core device, navigate past the point of obstruction, and reattach to the device once their intended location has been reached. The end result would effectively be the same. Whether by accident or intentional, the core smart medical device becomes separated from one or more subcomponents, and then reassembles itself at a downstream location, where it becomes permanently positioned and functional.

Along the same lines, the present invention's ability to detach and reattach subcomponents of a smart device with autonomous capabilities can also be used when an individual device subcomponent becomes non-functional. Using the same exemplary embodiment of the smart cardiac pacemaker, suppose the battery used for power supply was to cease functioning, which effectively makes the cardiac pacemaker non-operational. The internal quality control features of the program of the smart device would identify this deficiency and the program would issue an alert to authorized end-users of this issue via electronic means (i.e., alarm signal, email, fax, text etc.).

In one embodiment, if the device component housing the battery has its own internal autonomous navigation capabilities along with the ability to detach itself from the core device, the program could command that it effectively separate itself (see FIG. 5, for example) and autonomously travel to an extraction site or a location in which another smart device could perform a battery replacement. In either scenario, either the new and/or replaced battery contained within an autonomous subcomponent could then return to the original location under program instruction, reattach itself to the core pacemaker, become activated, and continue operation. This would create the ability for damaged and/or non-functioning device subcomponents to be replaced and/or fixed in vivo, without the requirement for surgical extraction and replacement.

E. Automated Positional Change

In vivo medical devices are subjected to continuous voluntary and involuntary motion and/or movement, which inevitably results in positional change of varying degrees. Physiologic movement such as blood flow, air flow, and smooth muscle contraction can cause movement of devices, as will non-physiologic movements which occur in everyday life. While the degree to which these pressures cause device movement are often unpredictable, they inevitably increase with the duration of the device placement.

As a result of these well documented movements, healthcare providers will often monitor device positioning through conventional medical imaging technologies, such as radiography, ultrasound, and computed tomography (CT). But these surveillance techniques have a number of associated drawbacks including (but are not limited to) expense, radiation exposure, delayed diagnosis, and delayed intervention. In the latter case, intermittent medical imaging will often miss substantive device positional change which can affect performance and have associated iatrogenic complications.

An example of how conventional medical imaging is traditionally used is in the intensive care unit (ICU), where patients are routinely subjected to daily portable x-rays to monitor the positioning of support lines and tubes including (but are not limited to) thoracostomy tubes, Swan-Ganz (SG) catheters, central venous catheters, endotracheal tubes, and nasogastric tubes. Despite daily monitoring, however, it is fairly common for clinically significant device positional change to take place between daily x-rays, which if undetected can profoundly affect clinical status.

Examples of such impactful positional changes include (but are not limited to) advancement of the endotracheal tube into the right mainstem bronchus (causing atelectasis and collapse of the left lung), retraction of the nasogastric tube into the esophagus (causing gastroesophageal reflux and/or aspiration into the lung), advancement of the SG catheter into a distal pulmonary artery branch (causing vascular occlusion and pulmonary infarction), and retraction of the thoracostomy tube (causing expansion of a pneumothorax and/or air leak into the chest wall).

Further, there remains the significant limitation of time delays between the time a clinician is notified and the time remedial intervention takes place. Due to existing time pressures, work overloads, and human error, it would not be unusual for a diagnosed mispositioned medical device to be temporarily ignored, resulting in an adverse clinical action.

The present invention solves the above problems and provides a smart device with continuous device monitoring and diagnosis features, including real-time and immediate intervention at the point of care, which could also be independent of human involvement. The present invention provides a combination of continuous anatomic visualization and mapping, temporal analysis, and self-directed autonomous smart device navigation and positional self-correction.

In one embodiment, each time a smart medical device is inserted, its desired destination is programmed into the smart device computer system, which in turn may or not be synchronized with anatomic visualization and mapping data. In addition to the desired anatomic positioning of the smart device, an additional input is provided which quantifies the degree of acceptable positioning variability and directionality.

In an exemplary embodiment, suppose an endotracheal tube is being inserted into a patient for the purpose of airway control and ventilation. The desired anatomic position is 3 cm above the carina with an acceptable variability of +/−3 cm in a cephalad direction (towards the head) and +/−2 cm in a caudad direction (towards the feet). While this total acceptable positional variability is 6 cm, it is not symmetric, for the simple reason that 3 cm of distal migration of the endotracheal tube would result in entry into the right mainstem bronchus, which would constitute a serious complication.

In the exemplary embodiment, the autonomous endotracheal tube is inserted and arrives at its intended destination 3 cm above the carina using the present invention's navigational features. Once the acceptable variability in device position has been verified by the program, a retractable device anchoring device is deployed from the smart device by the program, the anchoring device which serves to minimize device movement. A number of in vivo device-anchoring options may be utilized including (but are not limited to) balloons, sutures, screws, coils, struts, and biocompatible chemical adhesives (see FIG. 2, for example). The point of emphasis is that whatever anchoring device is deployed, it has the capability of being portable and retractable, thereby allowing the anchoring device to be readily withdrawn and/or redeployed at any point in time.

In the incorporated patents/applications, a methodology was described for creation of a 4-D visualization mapping technique, which when applied to autonomous smart medical devices, provides continuous analysis of smart device in vivo positioning. Alternatively, in conventional medical imaging technologies, the resulting imaging data could provide the program of the smart device intermittent feedback so as to provide updated positional information. Regardless of the manner in which updated anatomic data is collected, the smart device may receive periodic or continuous updates as to its anatomic positioning and any deviation which may have occurred relative to its original destination.

In the exemplary embodiment, with this updated positioning data (and ability to correlate with adjacent anatomy and/or pathology), the program of the smart medical device can now determine whether smart device repositioning is required for optimal performance. Suppose in the example of the smart endotracheal tube, the device has migrated 12 mm from its original location and is now positioned 1.8 cm (or 18 mm) above the carina. Referring back to the acceptable positional variability, this new device position remains in an acceptable location and no proactive intervention is required. If, however, the program reports that the device has migrated 22 mm (instead of 12 mm) from the collected data, the program will determine that the new position lies outside of the acceptable positional range and will indeed require intervention and repositioning (i.e., device positional self-correction).

In this exemplary embodiment, once this positional self-correction feature is activated by the program, the anchoring device is retracted by the program, which allows unimpeded movement of the smart device. The distance and direction between the optimal device location and current location (i.e., anatomic positioning) is calculated by the program and the positional change coordinates are inputted by the program into the smart device autonomous navigational controls. The activated navigational controls now cause the smart device to actively navigate to the desired location. Once completed, a verification feature is activated by the program, which determines the current smart device location and correlates this positioning with the intended location. If another positional self-correction is required, the exercise is repeated, until the new smart device location has been confirmed as both accurate and acceptable. At this point in time, the anchoring device is redeployed, and the smart device is securely positioned.

In one embodiment, as noted above, the degree of smart device autonomous navigation may be variable. At the one extreme, the smart device may be fully self-navigational, while at the other extreme, the device may require external supervision and input by an authorized end-user. In between, there are various degrees of device independence and external assistance.

In one exemplary embodiment, with respect to device positional change, while most device positional changes are minor in nature, on occasion a dramatic positional change may occur, which may not only affect functioning of the device but also incur potential damage to the device as well as the host patient. In this exemplary embodiment, an inferior vena cava (IVC) filter has been dislodged from its normal stationary positioning within the inferior cava (based upon deployment of struts which are embedded in the IVC walls) and is now mobile and freely circulating in the bloodstream. With the struts extending outwards, direct vascular injury and bleeding are primary concerns.

In conventional practice, the only practical solution is retrieval of the device, which can be performed by either surgery or through percutaneous insertion of a large bore catheter with retrieval capabilities.

However, with the present invention, two other options become available. In one embodiment, there can be an in vivo retrieval of the IVC filter by a smart device which can use its internal tracking program function to locate the renegade IVC filter and deliver it to a predetermined site for extraction. In another embodiment, and possibly more preferably, the program can activate the autonomous navigation function of the IVC filter after retracting the anchoring struts, and have the filter self-navigate back to its intended destination site within the inferior vena cava. Once that has been performed and the position verified by the program, the struts can be re-deployed and the device anchored once again in its proper position. While the degree of positional change varies greatly in these two examples, the functionality remains the same, in that smart medical devices utilize their autonomous navigation capabilities for positional self-correction.

F. On/Off Switch

In one embodiment, a safety/security feature of the present invention includes the ability to turn on and off the autonomous navigation. In order to ensure safety and security of the smart device, an on/off switch can be embedded within the smart device, which ensures that the autonomous navigation function is only activated by the program when appropriate and cannot be under the control of an outside unauthorized third party. After all, if the smart device has its own internal computer which communicates with other devices through wireless transmission, the possibility of hacking of the device computer must always be considered and protected against.

In one embodiment, while a number of potential security features can be incorporated into the smart device architecture and operability, the simplest method is to incorporate an on/off switch which requires an authentication protocol for activation. Once the inputted security code has been verified by the program, the autonomous navigation feature can be turned on, thereby allowing the smart device to embark on its intended mission or self-correct its position, on an as-needed basis. This as well as numerous other safety and security features will be described in detail under the Safety and Security section below.

G. Defining Device Position and Variability Limits

In one embodiment, the determination of optimal device positioning and acceptable positional variability can be defined in a variety of ways including (but are not limited to) best clinical practice guidelines, authorized end-user input, artificial intelligence (i.e., algorithms created through data mining of electronic medical records), medical device manufacturer recommendations, and correlation with patient-specific anatomy. Once the device positional protocols are established, they can be modified and/or refined at any point in time, based upon individual and collective patient experience, anatomic variability, and underlying pathology.

In one embodiment, the specific clinical context in which the smart device is being used has ramifications on positioning which may supersede the conventional positional parameters. For example, suppose a given smart device is being used in a multi-functional capacity. In one use case, a smart device with multiple drug reservoirs (which we will denote as the drug delivery device) is being used to deliver different pharmacologic agents to two different in vivo infusion catheters (denoted as recipient devices). In the first delivery, the recipient device has an embedded reservoir measuring one (1) cm in diameter, which means the delivery device needs to position itself with a variable distance of 5 mm from the epicenter of the recipient reservoir.

In one embodiment, the second recipient device contains a recipient reservoir measuring only 5 mm in diameter, which means the positioning of the needle from the delivery device needs to be within a distance of 2.5 mm from the epicenter of the drug recipient reservoir. In addition, the configurations of the first and second recipient devices are significantly different from one another, with the first assuming a linear configuration and the second a trapezoid configuration.

As a result, the manner in which the delivery device must orient itself relative to each individual recipient device is different and needs to be specifically clarified by the program and/or user in the positioning instructions provided to the delivery device at the time of its implementation.

To add one more complexity to the illustration, in one embodiment, suppose in the process of delivering the drug to the first recipient device, it is determined that the first recipient reservoir has a defect in a small portion of its surface (to the left of midline), which effectively reduces the target size of the reservoir from 10 mm to 6 mm. Once this has been identified by the program, the positioning and acceptable variability changes are inputted into the patient-specific database, so that future drug deliveries can be analyzed and anticipated by the program and necessary adjustments can be made by the program.

In another exemplary embodiment, suppose an individual patient's hepatic arterial anatomy is different from the norm (i.e., anatomic variation), with the right hepatic arterial branch arising 2 cm proximal to its normal location. If a smart device is being used to deliver a drug to the liver, it might normally be positioned 5 cm from the origin of the hepatic artery. However, in this particular case, the ideal positioning of the drug delivery device would be only 2.5 cm from the origin of the hepatic artery, in order to accommodate for the atypical proximal branching of the right hepatic artery. The end result is that smart device positioning may sometimes be patient and/or context specific, depending upon individual patient anatomy, clinical context, and specific device configuration.

H. Security and Safety Features of Autonomous Smart Devices

Safety and security features of the present invention includes:
1. End-user authorization and verification.
2. Hierarchical privileges for authorized end-users.
3. Turning “on” and “off” various smart device functions (particularly autonomous self-navigation).
4. Selective activation of individual smart devices functions/miniaturized devices.
5. Coordination, communication, and data sharing between individual smart devices in order to act collectively.
6. Automated alerts and notifications via electronic methods.
7. Emergency power “off” mode and extraction from host patient.
8. Redefining smart device activities and guidelines (in vivo and in real time).
9. Anti-hacking features.
10. Changing of data transmission and communication protocols (or technologies).

The fact that autonomous medical devices operate through computer networks puts them at a number of potential security risks, with hacking of primary concern. A few points of emphasis on cybersecurity are made below, which specifically relate to in vivo autonomous smart medical devices. However, the present invention envisions leveraging existing and future cybersecurity technology into its implementation.

In one embodiment, since various degrees of autonomy exist, human operators may often directly interface and communicate with the smart medical devices. As a result, the program includes authentication and authorization protocols which are integrated into smart device operability, in order to safeguard against non-authorized individuals from exerting influence on smart medical device operation. A number of well-documented technologies are readily available for such end-user authentication and authorization including (but are not limited to) single factor authentication (e.g., passwords), two-factor authentication (e.g., smart phone authorization codes), and biometric multi-factor authentication. A wide array of available biometric technologies is currently available (e.g., fingerprints, facial recognition, voice analysis, iris scanning, DNA, and vascular flow patterns), any one of which can be integrated into the invention for security purposes.

In one embodiment, it is not just humans which must undergo authentication and authorization in the application and use of smart medical devices, but also the various computer systems that run them. Whether these computer systems reside internally or externally, authentication is required in order to enable communication between these computers, since after all they are the primary parties responsible for autonomous medical device functioning. Based upon each individual computer's “signal transmission profile”, an authentication and authorization protocol can be searched by the program and/or user to ensure that the respective computers have the required clearance and permission for inter-operability.

In one embodiment, since autonomous smart devices often contain numerous miniaturized sub-components; each individual sub-component of device may have its own unique “signal transmission profile” as well. As a result, an individual host can be identified by the signal transmission profiles of the in vivo smart device in tow and/or its individual subcomponents. This security feature becomes important when certain end-users may be authorized to transmit and receive data from some smart device subcomponents and not others.

In one exemplary embodiment, suppose an infectious disease physician is authorized to obtain data from a specific biosensor contained within a smart device for chemical assays related to infection. But contained within the same smart device are biosensors which track heart rate and rhythm, which are outside of the purview of the infectious disease physician, but important data for a cardiologist. The net result is that a given smart device may contain numerous miniaturized devices, each of which has its own security features, which can be independent and separate from the device in toto, and from its other subcomponents.

In one embodiment, as higher degrees of authorization are required by the program for highly important and secure functions, the number and complexity of the required signal transmission profiles may become greater (in order to enhance security). In the same manner in which a cipher continuously changes its signal codes, the same is performed by the program for a given smart medical device. The key for authentication and authorization is to have the ability of the program to search the individual patient, computer, and/or medical device database; so as to identify all potential signal transmission profiles available at any single point in time.

Regardless of whether this authentication and authorization protocol is being applied to humans or computers, in one embodiment, all requested transactions can be recorded by the program in a separate database for auditing and analysis. This becomes an important security feature, for the program can then identify patterns of unwarranted access and/or data breaches. In the event that multiple unsuccessful attempts were recorded by the program in the database, the program could initiate an automated lockdown along with sending an automated alert via electronic methods to security personnel.

In one embodiment, the present invention would have the program initiate an emergency override, in the event that an unauthorized end-user was somehow able to gain access to the smart medical device. Since all communications with smart medical devices and corresponding computers are recorded and analyzed by the program, in the event that an unexpected and/or unusual communication was to take place, an automated escalation pathway would be implemented by the program, in order to ensure that appropriate safety and security measures were in place. The parties being notified by the program in such a case would be determined on the basis of the specific type of smart medical device, the clinical context in which it operates, and the nature of the communication.

In one exemplary embodiment of the automated notification and escalation pathway of the present invention, an acute cerebral infarct due to right middle cerebral artery thrombus requires the coordinated effort of multiple smart medical devices for treatment. Since time is of the essence in order to prevent irreversible neuronal death, it is imperative that blood flow to the occluded right cerebral artery be restored immediately. In order to accomplish this task, three smart medical devices are tasked by the program and/or user, including one smart device tasked with infusion of a thrombolytic agent, one tasked with physical dissolution of the thrombus, and one containing a filter device for entrapment of downstream thrombus fragments. Upon arrival at the destination site, the first catheter begins infusion of the thrombolytic agent; but before the second smart device can begin physical dissolution of the remaining thrombus, it must ensure that the filtering device has been deployed, in order to safeguard against debris being released downstream and potentially producing distal emboli and occlusion.

In the absence of the third device, in this exemplary embodiment, the program of the second smart device sends a communication to the third smart device to inquire as to its position and estimated arrival time. When no response is received by the program of the second smart device, the second smart device sends a message to the external computer system (i.e., computer system 104), alerting the failure of the third smart device. At this time, it must be determined by the program and/or user whether a new smart device is dispatched, whether the third smart device is to be deactivated, or whether the third smart device is in need of repair. In addition to becoming a safety issue, the possibility of a security failure must also be considered by the program and/or user.

In the exemplary embodiment, an audit of all communications to and from the third smart device is performed by the program at the external computer, with the goal of determining the cause of the third smart device's failure. Since the third smart device is no longer actively receiving or transmitting communication, it is determined by the program that the best course of action is to deactivate the third smart device and facilitate its extraction from the host. This will remove any safety and/or security concerns, while allowing a newly inserted device with similar functionality to be dispatched to the occlusion site and assist the second smart device in performing its function.

However, in another embodiment, where the third smart device did indeed arrive at the destination site but failed to deploy the filter device, since communication between the second and third smart devices are important to a successful outcome, a safety feature is implemented by the program to ensure that both smart devices are in proper positioning and the designated functionality has been tested and is fully activated before the assigned task is begun. In this alternative exemplary embodiment, that requires the second smart device to test its mechanical drill (which will be responsible for thrombus dissolution) to ensure it is properly working, while the third smart device must test its filter to ensure it too is functioning properly. Once the perfunctory quality testing has been implemented by the program, and completed, and the devices are properly positioned, communication takes place between the two smart devices to verify to one another that testing is complete, the smart devices have placed themselves in the correct positions, and the task can now begin.

In one embodiment, in the absence of receiving such a validation signal from the third smart device (or vice versa), the second smart device cannot begin its operation, since doing so could result in fragments of the broken thrombus to embolize. As a result, the program of the second smart device sends an alert to the external computer system, notifying of the failed quality control and request for further investigation.

I. Software Updates and Modifications

In one embodiment, remote access by authorized computer systems provides the ability to upgrade software on an as-needed basis. This provides a relatively easy method for fixes to software malfunctions, addition of new security and safety features, and expansion of functionality. Since all computer-based access is automatically recorded by the program in a database, a centralized (e.g., cloud-based) permanent record is readily available for routine or emergent audits, in keeping with security and safety guidelines.

In one embodiment, software upgrades and modifications are important to all in vivo smart devices, regardless of their specific function and technical components. In the event that some smart devices do not possess compatible software, this may hinder inter-device communication, data sharing and functionality. In addition to upgrades through wireless transmission, physical add-ons of microcomputers may be required, which can take place through specialized smart devices, which may serve to implant the new and more sophisticated microcomputers into a designated port on the smart device.

In one embodiment, each individual miniaturized device which is embedded within the smart device may contain its own internal computer and operating system, which has the capability of internal upgrades separate from the primary computer system of the larger all-inclusive smart device, or the external computer system. This provides a method for the numerous miniaturized components in different smart devices to operate synergistically with one another.

In an exemplary embodiment, suppose a specific type of biosensor requires an upgrade to enhance detection of a specific biochemical compound. These specific types of individual biosensors may be contained within a wide variety of different smart devices. By having the ability to upgrade software for each individual biosensor, the entire class of biosensors can function in concert with one another, irrespective of the specific type of medical device in which they are embedded.

In one embodiment, when a security threat is encountered, the individual operating systems of the affected smart devices and/or subcomponents can be remotely shut down by the program of the external computer system. If and when the threat is aborted and/or nullified, the operating system(s) can be remotely turned back on, restoring function. This provides a method for containing and limiting the spread of security threats, while maintaining function of the other non-threatened components within an individual smart device.

J. Quality Assurance (QA) and Quality Control (QC)

In one embodiment, routine testing is required for all smart medical devices and their individual subcomponents in order to ensure they are operational, accurate, secure, and safe. In addition to the QC testing of each technical component, routine testing is also performed on the communication systems, which are also important to device operation and inter-device coordination.

In one embodiment, all QA and QC testing results can be automatically recorded by the program in a QA/QC database (i.e., internal and/or external) for review and analysis. Whenever routine testing identifies a potential deficiency as noted by the program, an automated escalation pathway can be triggered by the program, which ensures that the smart device and/or subcomponents of concern are removed from routine operation, until the deficiency in question has been satisfactorily addressed.

In one embodiment, where simple shut down of the involved component(s) and/or device is insufficient, extraction of the smart device may be required in order to ensure that the host patient and/or other smart devices are not adversely affected.

In one embodiment, with respect to the QA/QC testing and repair process, is the ability of the program to initiate repairs and/or replace the involved subcomponents or components of the smart device. In such a case, designated repair smart devices may be dispatched by the program to the location of the smart device in disrepair, where they can replace and/or repair the deficient component in question. Once the repair or replacement has been completed, remote QA/QC testing can be performed by the program to assess whether the operation has been successful. If the program determines it has, the smart device and/or subcomponent of concern can be recommissioned and restored to active duty by the program. If the program determines it is unsuccessful, the component and/or device will remain out of commission and/or extracted if necessary.

In one embodiment, since inter-network communication is important to smart device performance, the program records and analyzes all communications which occur at or between smart medical devices and their subcomponents in a database (e.g., internal database 109, external computer system database 118, or external database (not shown), etc.). Since each device and its subcomponents have their own unique signal (transmission) profile, the source and identity of all communications can be readily identified, with a number of recorded communication metrics including (but are not limited to) identity of the device, its location in the host, nature of the communication, its duration, time, frequency, and subsequent actions taken. In the event that a communication of concern is identified, the device(s) in question can be proactively monitored by the program and intervention takes place when indicated.

In one embodiment, the present invention can be used to address a lack of communication, which may cause a point of concern. As one exemplary embodiment, suppose a multi-device action is planned where multiple smart devices are acting in concert with one another in order to facilitate a complex action. In this exemplary embodiment, a patient with an acute stroke due to acute occlusive thrombus in the middle cerebral artery is being treated through a multi-smart device intervention. In this planned intervention, one smart device is designated to locally infuse a thrombolytic agent, a second device is designated to follow with a drilling device, while a third device deploys an umbrella to trap any small thrombus fragments. The collective action of the three devices aims to reduce the thrombus burden, so as to restore blood flow to the area of acute infarction before irreversible neural injury occurs.

In one embodiment, in order to work properly it is imperative that the drilling and umbrella devices act in a coordinated fashion, so that no thrombus fragments are allowed to pass into distal vessels and cause downstream occlusion. As a result, the communication between these two devices is important to ensure clinical success and avoid an iatrogenic adverse outcome.

In this exemplary embodiment, suppose the communication initiated by the drilling device is not verified and responded to by the umbrella device. Second and third communication attempts also fail, resulting in cancellation of the proposed action by the program. The infusion device proceeds as planned, since it operates independently and does not require assistance from another smart device to complete its task. But in the case of the drilling and umbrella devices, the task requires coordination between the two smart devices, so if either is non-operational or they are unable to communicate with one another, then the task cannot be safely performed. In such a scenario where communication is incomplete or unsuccessful, the impaired umbrella device is recalled by the program and a new umbrella device is dispatched. Once this has been completed and the communication signals are transmitted and verified by the program, the operation can recommence.

K. “Break Glass” Feature

Continuing this previous exemplary embodiment, suppose the drilling device fails to recognize the lack of responsiveness on the part of the faulty umbrella device and begins its drilling operation. This will effectively result in numerous small thrombus fragments being released into the cerebral artery which would likely lodge in distal cerebral artery branches and cause multiple new brain infarcts. In such a scenario, the only way to prevent a catastrophe would be to emergently turn off the drilling smart device. The program allows this emergent intervention under a so-called “break glass” feature.

The “break glass” feature of the present invention is primarily designed to serve as a safeguard for an emergent high security and/or safety situation, which could entail severe damage to health or even death, if left unattended. Under normal circumstances, a well-defined security protocol establishes chain of command for intervention. In this chain of command security protocol, a well-defined hierarchy is established for defining what parties have the power to intervene in smart medical device actions. A number of variables are defined including (but are not limited to) the specific type of smart device (or subcomponent), its location, the clinical context in which it operates, the target destination, other smart devices in which it interacts, and the scope of operation.

While the conventional security and safety protocol is designed to address most issues of concern, the possibility of a life-threatening emergency requiring immediate action may occur, which is so time sensitive that the delay associated with routine security protocols would prove to be costly. In such a truly emergent situation, the “break glass” feature of the present invention provides a mechanism for immediate intervention. One consideration is that the program would override or circumvent the security protocols in place, which could theoretically expose the system to malicious activity.

In one embodiment, if and when the “break glass” feature is deployed by the program, a series of urgent alerts would automatically be transmitted via electronic means (i.e., text, email, fax, etc.) to all authorized parties, with the program-verified corresponding high security clearance, notifying them of a potential security breach. In turn, any of these notified parties would have the ability to be rapidly authenticated and given access to the smart devices in question by the program. These individuals would then have the capability of overriding or modifying the “break glass” command, as clinically indicated.

As is the case with all other data inputs, the corresponding data is automatically recorded by the program in the operational database and amenable to both computerized and human audits and analyses.

L. Hierarchical Privileges

Regardless of whether data input or output is involving humans or computers, the sharing of data/information requires a well-defined delineation of privileges. As is the case with conventional clinical practice of medicine, privileges to sensitive data must be narrowly defined, in accordance with the profile of the involved parties. The same holds true for the ability to access and input data relating to smart medical devices.

In the exemplary embodiment of a semi-autonomous smart medical device, a human operator may be tasked with supervising and assisting with smart device navigation. But in order to ensure that the operator has the appropriate training and clearance, each individual interacting with smart devices must first be properly vetted and assigned specific privileges in accordance with what data they are privy to and what data and associated actions they can be associated with.

A number of variables will be considered in the definition of these privileges including (but are not limited to) the specific type of smart medical device, the clinical context in which it is being used, the anatomic location in which it travels, other medical devices in which it interacts with, the identity of the host patient, and the subcomponents contained within the smart device. In some instances, privileges for an authorized operator may be individualized for some subcomponents within a given smart device and not others.

In one embodiment, a smart medical device with an embedded miniaturized surgical instrument may have privileges assigned to a surgeon but not a cardiologist, whereas an embedded electrophysiologic sensor within the same device may have assigned privileges to a cardiologist, but not a surgeon. At the same time, the navigational system within the same smart device may have privileges assigned to a biochemical engineer, who does not possess similar privileges to the surgical or electrical biosensor. In this manner, privileges related to a single smart medical device may be assigned to individual subcomponents or operating systems at the exclusion of others. A select few, may have more expansive or even complete privileges for a smart medical device (i.e., smart device super-users), and these select few are often the ones with the highest security clearance allowing for the “break glass” application.

In one embodiment, in a similar manner, hierarchical privileges and associated data accessibility can also be assigned by the program to other medical devices and/or computers. This defines how individual smart medical devices and/or their subcomponents may function in tandem with other smart medical devices. In a previously cited exemplary embodiment use case, multiple smart devices working in concert with one another were assigned to the task of removing occlusive thrombus from an occluded cerebral artery. One smart device was responsible for drilling thrombus, another with infusing a thrombolytic agent, and another deploying an umbrella to trap small thrombus fragments. The ability for these individual smart devices and their subcomponents to interact and communicate with one another is in part defined by their privileges, which define communication protocols and data accessibility between the computers within each individual smart medical device.

In one embodiment, it is important that these privileges may be of variable duration, so as to allow an authorized end-user a defined time period in which data is accessible. This serves as a security feature limiting both the time and extent to which a given human or computer may have access to a given smart medical device.

In one embodiment, all interactions between authorized end-users (both human and computers) and smart devices can be recorded by the program into a database for analysis. In the event that a given interaction was deemed to be improper (i.e., safety and/or security risk), the associated privileges can be modified (e.g., downgraded or terminated), based on the determined level of negative interaction by a team of clinical and technical experts.

In one embodiment, another security and safety feature which can be incorporated into the invention is blockchain technology, which can lead to the creation by the program of a virtual secure ledger for assignation and determination of smart medical device privileges, which cannot be readily altered. The incorporation of blockchain technology in the program of the present invention provides a shared immutable ledger that facilitates recording and tracking of data transactions and communications within the diverse smart medical device network.

M. On/Off Functionality

As mentioned above, the smart medical device safety and security of the present invention is, in part, related to the ability to shut down function on demand; thus, on/off functionality may be implemented at both the level of the entire smart device and its individual subcomponents. In some circumstances, on/off functionality may also be applied to multiple smart medical devices when working in concert with one another, as illustrated in the previous exemplary embodiment use case of the occluded cerebral artery. In that exemplary embodiment, malfunction on the part of the umbrella smart device (which serves to trap small thrombus fragments), will result in the program instructing simultaneous shut down of both the umbrella and drilling devices, which are dependent upon one another for safe operation.

In one embodiment, the idea of applying on/off group functionality is particularly relevant to nanobots and microbots (“bots”), which are often present in large numbers given their small size. These are in effect, also smart medical devices, but in miniaturized size. As a result, in vivo smart bots often function in groups, which can be collectively communicated with through the transmission and receipt of a unique signal frequency. In the event that a group of bots tasked with a specific task or function requires termination of a given task, a single command by an authorized end-user can trigger the “off” function contained within each bot, thereby immobilizing an entire group of bots, which can number in the hundreds, thousands, or even millions.

In one embodiment, this on/off functionality may also serve an important component in smart medical device quality control (QC). When routine testing is performed by the program of the various subcomponents contained within a given smart device, it may be determined that one or more of these components (as well as the entire device itself) is no longer properly functioning. In such a scenario, the “off” function can be activated by the program, rendering the individual subcomponent(s) or device(s) no longer active. If and when the subcomponent and/or device functionality is returned, the “on” function can be activated by the program, so that function is restored. Return of function can be as simple as recalibration or as extreme as replacement of the component in question.

In one embodiment, on/off functionality can also be used in the setting of preventative maintenance, where various components of a given smart device may require calibration, software updates, or repair. During the time period in which preventative maintenance is performed, the associated smart device and/or components are deactivated (i.e., turned off) by the program, and subsequently returned to action by the program once the maintenance is successfully completed. Automated notifications are made via electronic methods (i.e., text, email, fax, etc.) by the program whenever the “off” function is triggered, to alert all authorized parties that the smart device/subcomponent is not currently functional. All actions taken from the time of on/off activation can be recorded by the program in a database and audited for safety and security purposes.

In one embodiment, the previously described “break glass” function of the present invention represents an extreme case in which the “off” function is activated by the program on an emergency basis. In order to reactivate the smart device/component, a series of safety and security measures would be required by the program for reactivation of the device after the “break glass” feature has been deployed.

In one embodiment, on/off functionality need not be exclusively binary, but instead can be scalable. As an example, if a smart vascular catheter has been successfully positioned at its destination site within the superior vena cava, the navigation component of the smart device may no longer require complete activation by the program, but instead can be placed in a semi-active mode by the program. Using a scale of 0-9 for on/off functionality, where 0 is completely “off” and 9 is completely “on”, the navigation component of the smart device once it has been properly positioned, may now be reduced to 3 in an exemplary embodiment. This allows for energy conservation while also minimizing the degree of sensitivity, with regards to repositioning. If, in this exemplary embodiment, the device's position was to deviate by more than the (predetermined) allowable 5 cm, the navigation system would automatically be triggered to readjust device positioning, On the other hand, if the on/off functionality was set to a higher level of 6 (as opposed to 3), the positioning triggering mechanism may become activated by the program at a positional change of lesser magnitude (e.g., 3 cm, as opposed to 5 cm). Thus, the on/off functionality can be scalable on an as-needed basis.

In one embodiment, the activation or modification of the on/off functionality can be controlled by a variety of authorized sources, including (but are not limited to) the smart device program in question, another smart device program, human operator, external computer program, or database (by exceeding a predefined threshold).

In one embodiment, on/off functionality can also be controlled by a timer for a variable duration, in a manner analogous to a home smart device controlling lighting. In addition, on/off functionality can be automatically triggered by the program for changes in health status of the host patient. In one exemplary embodiment, suppose that biosensors embedded in a number of smart devices serve to measure cytokines in the bloodstream. Since these have remained non-measurable over a prolonged time period, the corresponding biosensors have been effectively turned “off”. However, if the host patient's health status was to suddenly change and a fever was detected, the corresponding cytokine biosensors could be automatically turned back “on” by the program, and they would now become fully activated. This illustrates the dynamic nature of smart device on/off functionality of the present invention, which can be modified by both manual or automated methods.

N. Inter-Device Communication

In one embodiment, in addition to having control over its own navigational system and ability for multifunctional autonomous operation, individual smart medical devices also possess the ability to communicate and directly influence operation of other smart medical devices, resulting in group coordinated activity. Using artificial intelligence and machine learning, in one embodiment, these devices can actively learn and adapt to one another's navigation, particularly when the activities they engage with one another in are repetitive in nature.

In one embodiment, using each individual smart device's ability to send and receive signals, smart devices can actively track one another's 4-D in vivo location and directional movements. In the event that a smart device's movement is perceived by the program to be contrary to programmed expectations, a warning signal can be transmitted by the program via electronic methods (i.e., fax, text, email, etc.), for heightened evaluation of the device in question. This serves as an added safety and security measure in the event of smart device malfunction or malevolent manipulation.

In one embodiment, all inter-device communications can be recorded by the program into a centralized or external database for analysis, creating an added security/safety feature, along with data to drive future software development and technology refinement.

a. Exemplary Embodiment Use Case:

To illustrate how inter-device communication and coordination can work, take the example of a patient with lower extremity deep venous thrombosis (DVT) and saddle pulmonary emboli (PE). In this clinical setting, two separate procedures will be described to address the lower extremity and pulmonary arterial thrombi.

In this exemplary embodiment, for treatment of the lower extremity, an inferior vena cava (IVC) filter is deployed from a single smart medical device, which acts to trap migrating thrombi originating from the lower extremity DVT. For treatment of the PE, a combination of smart medical devices is utilized. These include a device infusing a local thrombolytic agent to help dissolve the occluding embolus, which is later followed by a pair of smart devices acting in coordination with one another. In this exemplary embodiment, the first smart device will deploy a mechanical drill for breaking apart the remaining thrombus, while the second device will deploy an umbrella a few centimeters away from the drilling device, which serves to catch small embolic fragments and preventing them from travelling downstream, where they could obstruct distal pulmonary arterial branches. It is important that these two devices operate in a synchronous fashion to one another, to avoid iatrogenic complications.

Thus, in this exemplary embodiment, the collective operation involves four separate smart medical devices in total, two of which will act independently and two which act in a well-orchestrated and coordinated fashion. For the latter two which act in concert with one another, inter-device communication is important to achieving a successful clinical outcome. For the other two devices, inter-device communication is helpful, but the timing and criticality of this communication is of lesser importance.

In this exemplary embodiment, in the first action taken, the smart device deploying the IVC filter is dispatched and once the program has successfully verified its correct positioning, the program releases the IVC filter. Subsequently, the sensors contained within the IVC filter provide data regarding their individual positioning relative to the IVC walls, which is recorded and verified by the program, and the program tests each individual strut for proper functionality. Once this internal test is completed by the program, the program has the smart device deliver a test injection of water-soluble particles to test the positioning and trapping function of the IVC filter. If the test is satisfactory according to the program's analysis with respect to predetermined parameters, the smart device returns to its baseline anatomic poisoning, using its autonomous navigation system.

In this exemplary embodiment, now that the lower extremity DVT has been satisfactorily addressed, the next (and clinically more important) order of business is to deal with the large centrally localized PE. Since this involves the deployment of three separate smart devices in a two-stage procedure, the timing and order of smart device deployment is important.

In the exemplary embodiment, the first device deployed by the program is the device tasked with infusion of the thrombolytic agent, of which anatomic positioning is very important to avoid systemic complications. For this reason, navigation and precise positioning of the infusion port in direct proximity of the thrombus is important for optimizing clinical outcome. The manner in which this targeted navigation takes place has been described in detail elsewhere in the description of the present invention.

In this exemplary embodiment, after localized infusion of the thrombolytic agent has been completed, the remaining thrombus can be physically removed through the coordinated efforts of a plurality of smart medical devices containing a drilling apparatus and umbrella. In this exemplary embodiment, the drilling device is tasked by the program with physical breakup and suctioning of the thrombus, while the umbrella device is tasked by the program with catching all downstream debris which becomes detached from the primary thrombus and enters the bloodstream. If these debris fragments were not trapped by the umbrella device, they would pass into distal cerebral artery branches, occlude smaller vessels, and produce a series of infarcts. As a result, the coordinated efforts of the drilling device, suction apparatus, and umbrella are important to eradicate the thrombus and prevent strokes from occurring.

In one embodiment, while exact poisoning of each device and its subcomponents is important to operational success, of greater importance is the program coordination and timing of the devices, to ensure that each individual device and its subcomponents are synergistically functioning both independently and in concert with one another.

In one embodiment, the ability of individual smart devices and their subcomponents to communicate with one another provides an important tool for coordinated activities. As each individual device navigates to its intended anatomic position, the program sends a series of signals to update other (smart) devices and (internal or external) computer systems of its position. Once the smart device has successfully arrived at its intended destination, the program can signal the other smart devices, which in turn can communicate its position. Once all involved smart devices have reached their intended positions and have communicated with one another, testing of the devices and subcomponents can be performed by the program, prior to commencement of the procedure.

In one embodiment, in the event that a given smart device or its subcomponents are not properly positioned or functioning, the program will not allow the procedure to go ahead, until the requisite problem has been resolved via amelioration methods noted above, or by turning the system off etc.

In addition, in one embodiment, if any smart device does not properly communicate with its designated partners, the program will instruct the procedure to remain on hold. This inter-device communication provides an important safety feature to ensure that the coordinated actions of each device are verified before beginning the procedure.

In this exemplary embodiment, suppose the smart device containing the umbrella apparatus is not in the correct position, is not fully functional, or has failed to communicate with the drilling device. In any of these scenarios, the drilling device will not begin until it has received communication from the umbrella device and/or the central/external computer system that all required steps have been verified by the program and the procedure can proceed.

In one exemplary embodiment, suppose the drilling device violates the established protocol and begins operation without first receiving the required verification from the program. This could in effect create detached thrombus fragments from travelling distally and obstructing smaller downstream vessels.

In one embodiment, a number of safeguards exist for the program to prevent and/or limit the potential of such an adverse action. These include (but are not limited by) the following:
1. Automated termination in absence of corroborating signal from partnering smart device.
2. Automated termination upon receipt of distress signal from partnering smart device.
3. Termination upon receipt of signal from central computer monitoring communications.
4. Termination command from authorized human operator monitoring communications and smart device actions.
5. Activation of “break glass” feature causing immediate and irreversible shutdown.
6. Automated cessation of activity triggered by program auditing and analyzing inter-device communications.
7. Activation of intervention smart devices to minimize negative impact in the event of shutdown failure.

In one embodiment, the last item in which an intervention option is deployed could include a myriad of possibilities, in which specialized smart devices are deployed in an effort of damage control. In this specific example, one option may include the release of large numbers (i.e., thousands or millions) of specialized nanobots in the host patient, which possess the ability to release short distance and low frequency lasers into the nearby paths in which they travel.

In one embodiment, the lasers being emitted by these circulating nanobots are designed to ensure that they cause no damage to normal tissue they encounter but would serve to disintegrate any large particulate matter in their path. One of the highly specific applications of the present invention is the elimination of small thrombi and/or emboli which are freely circulating in the bloodstream, as in the case of this specific case. By injecting large numbers, the circulating nanobots will effectively create a continuous stream of lasers as they pass through the area of clinical concern.

In certain circumstances where their location is restricted, a specialized injection site may be required, in lieu of the normal introduction via the peripheral bloodstream. In this particular case, where the blood brain barrier may restrict nanobot entry into the cerebral arteries, an alternative intrathecal injection may be required.

In one embodiment, the net effect is that when an iatrogenic complication is identified related to smart medical device activity, other specialized smart medical devices may be deployed for treatment. Since every operation has the potential for an unplanned mishap, multifunctional smart medical devices can play a vital role in counteracting the negative impact. At the center of preventing such a mishap is inter-device communication of the present invention.

O. Smart Device Elimination and/or Extraction

From a practical perspective, the implementation of smart medical devices of the present invention, is not complete without an exit strategy, which can consist of either physiologic elimination from the host patient or physical extraction (see also section C.e. above).

In one embodiment, under normal circumstances, smart device removal from the host patient is an elective process, which may be triggered by a number of processes including (but are not limited to) smart device mechanical failure, completion of clinical task, smart device obsolescence, or requirement for smart device repair. In rare circumstances, the smart device retrieval may be the result of unexpected activity, and in such a case, an emergency evacuation is required, in order to alleviate any danger or adverse action.

In one embodiment, in addition to the “break glass” option which has been previously described, for immediate shutdown of a smart device under extreme circumstances, an additional safety and security feature of the present invention is a “self-destruction” option, in which an authorized operator can send out a signal and trigger an internal implosion device which causes the smart device to be destroyed, with minimal impact beyond the confines of the device.

In one embodiment, regardless of the mechanism of immobilization, a smart device which has been voluntarily or involuntarily decommissioned must have a mechanism in which it is removed from the host patient. A number of elimination and extraction methods exist for this purpose as previously described.

The primary difference between extraction and elimination is that extraction is physically supported by another entity, while elimination does not require physical assistance. In one embodiment, elimination can be the result of a smart device passing into a physiologic system for removal (e.g., gastrointestinal tract, urinary system, respiratory system). In one embodiment, while nanobots are small enough to also be transdermally eliminated via perspiration, larger smart devices can also be eliminated transdermally through activation of a boring device, which allows a puncture in the skin surface to be created in which the device can exit the host patient.

In one embodiment, extraction, on the other hand, requires physical assistance for device removal from the host. This assistance can be provided from other smart devices or authorized human operators. In addition, to extracting the smart device in its whole state, smart devices may also have the ability to be broken down into subcomponents, depending upon the device structure and composition. Some devices may be constructed in an articulated format, allowing the individual articulated components to be detached from the central core of the device, thereby allowing for extraction of multiple smaller parts. Other smart devices may have appendages (e.g., cardiac pacemaker), which provide a natural mechanism for disassembly prior to extraction. Lastly, smart devices can also be physically downsized or fragmented through controlled implosion, rendering it into multiple smaller pieces for easier extraction. Regardless of the strategy employed, extraction includes the program propelling or transporting the smart device in tow or in parts to a designated extraction site for final removal.

In one embodiment, the simplest method of extraction is via towing of one smart device by another. In the event that a smart device is disassembled or fragmented into multiple pieces, multiple smart devices may be required for extraction. Alternatively, in another embodiment, if a smart device is destroyed, resulting in multifocal debris and/or small components, one alternative strategy can be utilized such as a vacuum or filter equipped smart device or lasers for complete dissolution of the smaller fragments. The smart devices participating in these extraction techniques may do so autonomously (by program) or under the direction of an authorized human operator.

In one embodiment, when physical extraction requires minor surgery, the smart device can be navigated to a designated superficial location and an incision made by an authorized operator or robot for final removal of the smart device. When physiologic elimination occurs, the smart device in question can be captured by filtering the medium in which it passes (e.g., air, feces, urine).

Upon retrieval, the smart device and/or its subcomponents can be collected and subjected to additional testing on an as needed basis. In some circumstances, biological material has been collected and this can be retrieved from the storage device in which it was collected.

P. Anti-Hacking

In one embodiment, as is the case for any computerized system (and especially the case for in vivo medical devices), anti-hacking features are important to assure safety and security. As previously described, a number of technical solutions can be applied to the invention including (but are not limited to) encryption, blockchain, multi-party authentication, and biometrics.

In one embodiment, in the event that an unusual, unexpected, or unauthorized smart device action takes place, an automated alert would be triggered by the program which would notify authorized responsible parties via electronic methods for engagement and feedback. In addition, the operator currently tied to the actions of the smart device in question would be required to undergo reauthentication and verification by the program. In the event that they failed to adequately provide this and/or other authorized operators determine the action to be unsafe or contrary to the standard of care, then the smart device would be immediately cancelled from further action by the program and the operator privileges revoked.

As previously stated, in other embodiments, other security features such as “break glass” and/or smart device destruction can be deployed under extreme circumstances.

Q. Examples

In order to illustrate how the present invention works, take the example of a commonly encountered medical procedure, laparoscopic cholecystectomy, for the treatment of chronic cholecystitis in association with gallstones.

As is the case with any surgical procedure, post-operative complications are commonly encountered, and this is especially the case with laparoscopically performed procedures due to the fact that visualization of the operative field is less than that of conventional open surgery.

Shortly after completion of the procedure, which was lengthier than expected due to excessive inflammation in and around the diseased gall bladder, the patient began experiencing pain and her vital signs showed an increase in baseline heart rate (i.e., tachycardia). In response, the surgeon of record ordered blood work consisting of a complete cell count (CBC), which identified diminution in the red blood cell (RBC) count and hemoglobin, raising concern for acute post-operative bleeding.

Following the CBC results, the surgeon ordered an abdominal/pelvic CT scan, which demonstrated high attention fluid in the post-operative fossa as well as the dependent portion of the pelvis, which was suspicious for post-operative bleeding.

The patient was subsequently transfused with two units of blood and intravenous fluids, which stabilized her heart rate, which remained slightly higher than baseline. Repeat CBC and CT scan were ordered for follow-up 6 hours later. The repeat CBC continued to show decreased RBC and hemoglobin while the follow-up CT scan showed continued hemorrhagic fluid in the post-operative gall bladder fossa, along with increased high attenuation fluid in both the abdomen and pelvis, which was far more than visualized on the initial post-operative CT scan.

The surgeon considered reoperating on the patient, but decided against it, given the fact that she remained relatively stable. The surgeon reordered another two units of blood as well as fresh frozen plasma, hoping the degree of blood loss remained relatively minor and would terminate over time.

Unfortunately, the patient's condition continued to deteriorate and the surgeon was forced to reoperate eight (8) hours later. Given the patient's condition and previously failed laparoscopic procedure, the surgeon was now forced to perform an open emergent operation. At surgery, it was discovered that two of the surgical clips ligating the cystic artery had come loose, resulting in bleeding. The surgeon tied off the bleeding site and placed a surgical drain to remove any continued bleeding or oozing in the surgical bed. His plan was to repeat the blood work and CT scan to ensure active bleeding resolved post-operatively and pull the drain in a few days once the patient's clinical condition warranted it.

However, the diagnosis and treatment of this relatively commonly encountered complication of post-operative bleeding could be performed in a different manner, given the present invention and its various applications. While blood work and conventional medical imaging studies (e.g., CT) could remain diagnostic options, an alternative strategy that utilizes the present invention would include deploying smart medical devices in a variety of forms for diagnosis and/or treatment.

One such option could utilize the administration of smart nanobots or microbots with embedded biosensors, as described above and in the incorporated patents/applications, in which large numbers of circulating nanobots, in one embodiment, can detect a variety of biologic markers (i.e., biomarkers) in the bloodstream, including those related to active bleeding and blood breakdown products. The specific anatomic location at which these circulating nanobots detect these biomarkers can in turn be localized based on program analysis of the signal emitters and/or receivers contained within the nanobots. In one embodiment, the information derived from this smart nanobots/microbots can both localize the sites of active bleeding as well as quantify the extent of bleeding (in a manner far superior to conventional medical imaging technologies). Based on this information, a number of strategies utilizing smart medical devices can be employed for therapeutic response.

In one embodiment, the same nanobots are utilized which diagnosed and localized the active bleeding in a therapeutic manner, by coalescence of numerous nanobots into a cluster of macrobots, which can serve to physically occlude the actively bleeding blood vessels.

In another embodiment, these same nanobots could excrete a thrombogenic agent, whose release can be triggered by the program when the biosensors contained within the nanobots provide data to the program that confirm the local presence of bleeding biomarkers.

In another embodiment, a smart medical device strategy may include deployment of larger and more functionally complex smart medical devices to the active bleeding sites, where they could utilize a variety of strategies to reduce and/or cease active bleeding. These options could include (but are not limited to) local infusion of thrombogenic agents, microsurgery and/or ligation to repair the bleeding vessels, coil embolization, administration of gel foam or medical grade superglue.

Regardless of the strategy and specific type of in vivo smart medical device used, the common denominators would include the program performing localization of the active bleeding site(s), quantifying the extent and severity of bleeding, continuous tracking and interval change of active bleeding, anatomic guidance of smart devices to the bleeding sites, and continuous monitoring to determine treatment response.

In one embodiment, while external data (e.g., CT imaging data) can be used to assist in the diagnostic process, it is not necessary given the diagnostic ability of smart medical devices, which have a number of theoretical advantages over conventional imaging technologies as previously discussed.

Thus, based on the above, the various time delays in diagnosis and treatment associated with conventional medical practice could be reduced and/or eliminated using the present invention, where the program's ability to provide real-time and continuous diagnosis at a molecular level, and anatomically localize pathology on a more granular level, provide anatomic guidance for in vivo therapy, and for continuous monitoring treatment response, and to rapidly detect potential complications.

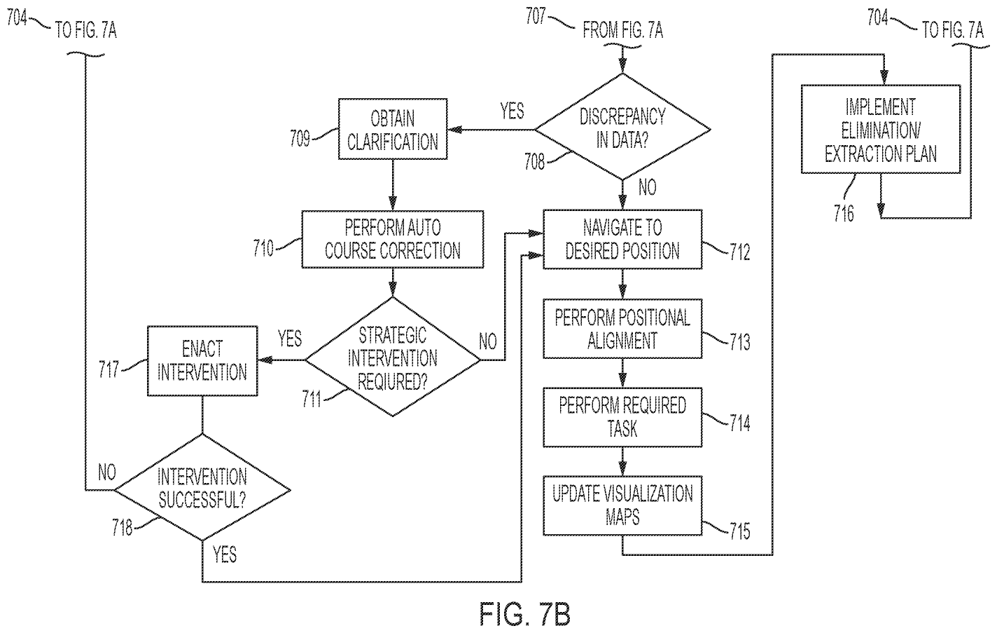

In one embodiment, the following is a representative list of method steps or actions for illustrative purposes (see FIGS. 7A and 7B):
1. Introduction of Microbots for diagnosis and/or treatment.
2. Nanobots introduced via injection of peripheral vein (e.g., antecubital fossa).
3. Preprogrammed nanobots begin circulating through bloodstream after QC tests are performed for calibration and function (step 701. FIG. 7A).
4. If tests are successful (step 702), the nanobots are activated and circulate (step 705), and signal emitters and/or receivers continuously provide locational data for external tracking by the program. If tests are not successful, remediation is applied (step 703) including software updates and other steps. If they continue to be unsuccessful, the device is retrieved (step 704) via elimination or extraction.
5. If the nanobots are successfully employed, embedded biosensors within nanobots continuously acquire real-time, in vivo biologic and positional data (step 706), and visualization maps are produced.
6. Real-time data derived from nanobots is transmitted via wireless technology to external or remote computers, and external data sources provide data to the nanobots and the datasets/maps are compared (step 707).
7. Data is recorded in the external database of a computer system and analyzed by the program, providing updates to anatomic and pathologic data records. If there is a discrepancy (step 708, FIG. 7B), clarification is obtained (step 709) which can result in automatic course correction among other remediations (step 710).
8. Artificial intelligence techniques can be applied by the program to assist in data analysis and strategic intervention (step 711). If strategic intervention is enacted (step 711), if it is successful, the nanobots will navigate to the desired position (step 712). If not (step 718), the device is retrieved (step 704).
9. Once at the desired position, positional alignment is implemented (step 713), and the task is performed (step 714). In this case, continuously acquired biosensor-derived data is used by the program to identify biologic markers for active bleeding.
10. Anatomic location of bleeding can be determined in a variety of ways:
a. Concomitant traditional medical imaging technologies (e.g., CT).
b. Nanobot-derived 4-D visualization maps created and updated by the program (step 715).
c. Location tracking of nanobots by the program at the time biosensor bleeding markers are detected.
11. Additional quantitative data derived from nanobots is used by the program to determine rate of active bleeding and measurements of accumulating hematoma at bleeding site.
12. Nanobots are instructed by the program to begin to coalesce to form macro-occlusive agents in an attempt to tamponade bleeding.
13. Continuous signals being emitted and received by nanobots are used by the program to determine specific location(s) of active bleeding and its severity.
14. Additional localization of the bleeding site can be facilitated by nanobots being instructed by the program to deposit localizing markers which possess the ability to send and receive signals. (This effectively serves as a beacon for future smart device navigation to the bleeding site.)
15. An additional method of anatomic localization may include the program instructing nanobots to anchor themselves at the bleeding site location.
16. Real-time continuous data analysis performed by the program of the external computer system which provides anatomic and pathologic updates, which can be used by the program and users for strategic planning and/or intervention.
17. In the event of active bleeding, additional nanobot intervention is required. In one embodiment, one such intervention is the introduction of specialized nanobots with vaso-occlusive capabilities. Examples may include nanobots coated with a vaso-occlusive substance or capable of an injection system for targeted release of chemical compounds.
18. These vaso-occlusive nanobots navigate to the active bleeding site using the signals emitted from deposited anatomic markers or anchored nanobots.
19. Upon arrival at the bleeding site, these specialized nanobots begin to intervene in accordance with their vaso-occlusive action as instructed by the program. In this example, specially coated nanobots aggregate at the bleeding site and/or release thromboplastin activators, which in turn facilitates coagulation and/or physical occlusion of the actively bleeding vessel(s).
20. Circulating nanobots with embedded biosensors continue to collect real-time data to which is used by the program to determine whether the bleeding continues, and if so the temporal change in the bleeding rate.
21. If active bleeding continues at an excessive rate, the program will determine whether additional smart device intervention is required.
22. Once the task has been completed, and no other tasks are required, the program implements elimination/extraction plan (step 716), which culminates in device retrieval (step 704).

R. Deployment of Macro Smart Devices

(Note that the preceding description of microbots is not a necessary prerequisite for the introduction of macroscopic smart medical devices, which in themselves can also serve as a “first line” of diagnosis and/or treatment and can readily operate independent of microbots. The previous description was merely provided to illustrate how microbots can be used in isolation or in combination with macroscopic smart medical devices.)

In one embodiment, the following is a representative list of method steps or actions for illustrative purposes of the deployment of (macro, but many steps are the same as with micro, see FIGS. 7A and 7B) smart devices:
1. Due to their larger size, introduction of macroscopic smart medical devices (which will heretofore be termed smart devices) requires a larger entry portal.
2. A large bore intravenous access port is inserted into the right femoral vein.
3. Smart device(s) are introduced via this femoral vein access port.
4. Upon in vivo introduction, quality control (QC) tests are performed on the device in tow as well as its miniaturized subcomponents, using the program, to ensure proper calibration and function.
5. Once the QC test is completed and successfully passed, the smart device navigation system is activated. (If device does not pass QC test, it must be fixed or retrieved).
6. The autonomous navigation system can be completely autonomous (i.e., self-navigating) or actively supervised by an authorized external and/or internal computer system or human operator.
7. The anatomic destination can be preprogrammed into the smart device operating system, directed by external signal transmission (e.g., signal emitting beacons deposited at the anatomic site of interest), and/or directed by external directives (e.g., human operator, CT imaging dataset).
8. As the device moves, embedded signal emitters and/or receivers of the smart device provides continuous real-time in vivo 3 and 4-D positional updates.
9. Wireless transmission allows for active and continuous communication between the smart device operating system and authorized computers, human operators, and/or other smart devices.
10. Anatomic positional data which is actively acquired by the smart device internal navigation system provides continuous updates which can be correlated by the program with external anatomic data sources (e.g., CT imaging dataset, nanobot derived 4-D visualization maps).
11. If and when a discrepancy exists between the real-time actively acquired smart device data and external data source, artificial (i.e., the program) (or human intelligence) is used for clarification.
12. Automated course correction feature of smart device navigation system (of the program) is activated by the program, and the program adjusts the course of the smart device as deemed appropriate.
13. When other in vivo medical devices are present, their navigational communications are monitored by the program and correlated with the course of the smart device of primary concern. In the event that an impediment exists, this information is relayed to the navigational system of the smart device by the program.
14. In the event that the program utilizes data from other smart devices, external anatomic data, or the smart device internal navigation system, and identifies an unanticipated navigational challenge, AI is used by the program to identify alternative options.
15. If and when a superior navigational path is identified by the program, the data is conveyed from the external or central computer system(s) (i.e., processor/program), for example (or other (i.e., distributed) computer system(s) including the smart device computer system), to the smart device operating unit.
16. Estimated travel times can be calculated by the program based upon real-time velocity data of the smart device, the current estimated distance to the destination site, and potential points of delay (in accordance with historical data as well as contemporaneous data of other actively navigating in vivo smart devices).
17. These travel time estimates are continuously updated and stored by the program in the database, based on real-time measurements for iterative refinement.
18. In the event that a time sensitive emergency was taking place, these time estimates can be used by the program to identify the optimal navigational course, selection of medical device(s), and communication with supporting medical devices.
19. While the smart device in question continues to navigate to its destination site, continuous real-time data is being recorded in the database and analyzed by the program to update the location, severity, and temporal change of active bleeding.
20. In the event that other supporting medical devices are required to fulfill the desired task, deployment of these additional smart devices can be initiated by the program or the user.
21. All involved and authorized devices communicate with one another and share their locational coordinates with the external or central database of the computer system(s), for program coordination of activity and modification of navigational course if needed.
22. As the primary smart device in question approaches the active bleeding site, local signal transmitters assist in the program fine tuning navigational guidance.
23. Final smart device positioning requires exact alignment of the smart device subcomponents (e.g., drug infusion port cauterization tool, microsurgical apparatus) with the exact location of bleeding. This is carried out by the program adjusting the positioning as the data comes in on the actual location of the device in comparison with the predetermined or desired position.
24. Once final positioning of the smart device and its principal subcomponents is established and verified by the program, the performance of the intended operation can begin.
25. The smart device communicates with the external or central computer system(s) (and other smart devices if applicable) for final authorization by the program.
26. Once this authorization is provided by the program, the program starts the operation of the smart device, with corresponding time stamped data recorded in both local (internal) and external or remote databases of the external computer system(s), or external storage databases.
27. In the event that other smart devices (i.e., partnering devices) are required for coordinated activity, the operation cannot begin until all involved devices have been verified and authorized by the program. This process requires a combination of active communication, location verification, and QC testing of important device components.
28. The program of the primary smart device now initiates its assigned task, which includes deployment of a local infusion of a chemical agent (e.g., vasoconstrictor, thromboplastin activator) from an internal storage of the smart device, via a needle or other deployment mechanism.
29. As the intervention proceeds, biosensors on the smart device provide data measurements that are continuously recorded by the program and stored internally in a database or emitted to an external database of an external computer system, for determining the clinical response of the intervention. These biosensors can be contained within the primary smart device or other neighboring smart devices.
30. As this real-time biosensor-derived data is recorded and analyzed by the program, the results are analyzed by the primary smart device (program) for the purpose of continuance, modification, or termination of the task being performed.
31. In this specific example, once the chemical agent infusion has been completed, active measurements are analyzed by the program and reveal a high rate of continuous active bleeding.
32. As a result, two additional smart devices are deployed by the program to the active bleeding site for alternative interventions (e.g., placement of embolization coils, microsurgical ligation of bleeding vessels).
33. Before departing the bleeding site, the primary (i.e., drug infusion) smart device deploys two small surgical clips, as instructed by the program, to serve as anatomic localizers with the ability to perform signal emission and receipt for anatomic guidance.
34. The two additional smart devices repeat the same navigational steps described for the primary smart device.
35. Upon their arrival, once communication, authorization, and QC testing has been completed by the program, the two additional smart devices are instructed by the program (primary device and/or external computer system) to perform their specific tasks.
36. Continuous measurements of active bleeding are performed and analyzed by the program to determine the intervention impact.
37. Once the tasks of each of these devices has been completed, it is determined by the program that active bleeding has been dramatically reduced, currently existing at a very low level.
38. Video components within these (or neighboring) smart devices (or circulating nanobots) survey the immediate area to provide updated anatomic and pathologic visualization data that is recorded and analyzed by the program.
39. These updated visualization maps created by the program, reveal the presence of a 7.5 cm hematoma at the bleeding site.
40. Before completing the operation, the program determines (via artificial and/or human intelligence) that placement of a drainage catheter is required to decompress the hematoma.
41. A smart device with the capability of deploying a drainage catheter is instructed by the program and/or human operator, to be sent to the site of hematoma, and follows the same steps previously described.
42. Once the drainage catheter has been properly positioned and verified by the program, the associated smart device is extracted at a predetermined extraction site.
43. The sites of prior bleeding and hematoma formation are continuously monitored by the program for signs of complication (e.g., new bleeding, infection).
44. In addition, the positioning of the drainage catheter relative to the hematoma is continuously monitored by the program.

45. On day three (3), it is determined by the program that the drainage catheter has migrated 3.5 cm from its original position and is now partly out of the hematoma cavity.
46. Positional data is communicated by the program to the navigational system of the drainage catheter (which has its own internal navigational operating system).
47. Once activated, the navigational system of the program that instructs the drainage catheter and repositions the drainage catheter into proper position.
48. This new position is assessed and determined by the program to be accurate, and the drainage catheter navigation system is turned off by the program.
49. Once the hematoma has resolved and the drainage catheter is no longer needed (based upon ongoing measurements and program analysis), the program instructs the drainage catheter it is no longer needed.
50. An extraction plan is devised by the program and/or human operator, and the navigational commands sent to the drainage catheter navigation operating system.

Although steps above are disclosed as being carried out autonomously or by the human operator, the method steps indicated could be carried out by either or both, depending on the programming.

As technology continues to follow the trends of automation, artificial intelligence, and miniaturization, a number of non-medical advances will eventually transition into medical practice. The autonomous devices of the present invention, when applied to medicine, will transform and eventually replace many of the surgical and interventional techniques currently in practice. Further, the real-time anatomic visualization maps of the present invention will augment guidance technologies contained within the medical devices.

The present invention creates technology capable of self-navigation with real-time adjustment of changing anatomy and pathology. The resulting autonomous smart medical devices can be applied to a wide variety of medical applications and disciplines and work in combination with one another in the performance of complex medical tasks.

By embedding signal emitters and/or receivers into smart medical devices, real-time tracking can be achieved, which provides a mechanism to monitor smart device activity and location in vivo, to ensure proper functioning and localization of the devices in question. In the event that safety and/or security concerns arise, a number of applications are incorporated into the technology to ensure optimization of host patient clinical outcomes.

The data derived from these smart medical device technologies can be automatically recorded, stored, and analyzed by the program of the present invention, for the purpose of determining best practices, which in turn can be applied to the creation of machine learning and artificial intelligence algorithms. The present invention provides independent medical technology which can rapidly adapt, iteratively learn, and synergistically function in vivo, with or without human operator input and guidance.

It should be emphasized that the above-described embodiments of the invention are merely possible examples of implementations set forth for a clear understanding of the principles of the invention. Variations and modifications may be made to the above-described embodiments of the invention without departing from the spirit and principles of the invention. All such modifications and variations are intended to be included herein within the scope of the invention and protected by the following claims.

What is claimed is:

1. A system which performs medical tasks in a body of a patient, comprising:
a medical device, including:
a signal emitter which emits energy in a form of a transmitted signal;
a signal receiver which receives transmitted energy as a received signal;
a plurality of sensors and/or detectors;
a propulsion mechanism and/or a steering mechanism;
an energy source; and
at least one processor which receives anatomic and positional data from said plurality of sensors and/or detectors and records said data in a database;
wherein said medical device is inserted in the patient and collects said anatomic and positional data in real-time from said plurality of sensors and/or detectors; and
wherein said at least one processor dynamically analyzes said anatomic and positional data on a continuous basis such that said medical device at least partially autonomously navigates to a desired position in the patient.

2. The system of claim 1, wherein said at least one processor is internal to said medical device, and further comprises:
an external signal receiver and/or transmitter which receives said transmitted signal from said medical device;
at least one external controller which receives said transmitted signal from said external signal receiver and/or transmitter and converts said transmitted signal into a standardized form of data; and
at least one external processor which receives said data from said external controller and records said data in a separate database.

3. The system of claim 1, wherein said propulsion system includes at least one of chemically powered motors, enzymatically powered motors, external field driven motors, internally mounted miniaturized electrodes, miniaturized electromagnetic pumps, or appendages.

4. The system of claim 2, further comprising:
an external energy charging source;
wherein said energy storage in said medical device can receive energy externally transmitted to said medical device from said external charging source; and
wherein said energy source is one of batteries, biofuel cells, thermoelectricity, piezoelectric generators, photovoltaic cells, or ultrasonic transducers.

5. The system of claim 1, further comprising:
an anchoring device attached to or disposed in said medical device, which anchors said medical device to said desired position.

6. The system of claim 1, wherein said medical device further comprises:
a lidar scanner which detects physical surroundings and distances from said medical device;
a plurality of inertial sensors which record movement of said medical device; and
at least one camera which provides visual tracking information to said medical device.

7. The system of claim 6, wherein said medical device further comprises:
a gyroscope which measures or maintains orientation and angular velocity of said medical device; and
a Global Positioning System (GPS) which provides the user with positioning, navigation and timing information of said medical device.

8. The system of claim 5, wherein said medical device further comprises:
a plurality of compartments containing at least one of:
a spring-actuated device, including at least one of a cauterization tool or a cutting tool;
a delivery device which delivers a product; or
an ejection device which ejects a product.

9. The system of claim 8, wherein said medical device under said at least partial autonomous navigation performs course corrections needed to stay on course to said desired position.

10. The system of claim 9, wherein said medical device deploys a marker from one of said plurality of compartments, said marker which emits signals processed by said processor of said medical device, which allows said medical device to position itself at said desired position.

11. The system of claim 10, wherein said anatomic and positional data collected by said medical device is provided to at least one other medical device by emitting signals from said signal emitter of said medical device, to facilitate autonomous navigation of said other medical devices to said desired position.

12. The system of claim 1, wherein said medical device includes a plurality of subcomponents attached to a main body, said plurality of subcomponents which can detach from said main body for individual navigation, and re-attach with said main body.

13. The system of claim 1, wherein said medical device is capable of merging with other medical devices and/or subcomponents into an aggregate medical device to increase functionality.

14. The system of claim 1, wherein said medical device is capable of at least one of collapsing in size by one of detaching one or more components or expanding in size by expanding on one or more components.

15. The system of claim 1, wherein said medical device is capable of being extracted from the body of the patient; and
wherein extraction occurs through one of towing said medical device by said at least one other medical device or through surgery.

16. The system of claim 1, wherein said autonomous navigation of said medical device is capable of being turned on or turned off.

17. The system of claim 1, further comprising:
a mechanism for immediate intervention in an emergency, said mechanism which circumvents security protocols; and
wherein a plurality of alerts is automatically transmitted by electronic methods to authorized parties.

18. The system of claim 16, wherein said medical device is turned off automatically in at least one of: an absence of a corroborating signal from a partnering medical device, upon receipt of a distress signal from said partnering medical device, upon receipt of a signal from said external processor monitoring communications from said medical device, upon command from an authorized user monitoring said communications, upon activation of said mechanism for immediate intervention, upon cessation of activity due to results of an audit and analysis of communications between said medical device and other medical devices, upon activation of intervention of other medical devices which act to minimize impact of a shutdown failure.

19. The system of claim 17, wherein said mechanism for immediate intervention includes self-destruction.

20. A method of performing medical tasks in a body of a patient, comprising:
receiving a plurality of signals from a plurality of sensors and/or detectors disposed in at least one medical device at a processor of said at least one medical device;
wherein said plurality of signals provide anatomic and positional data in real-time to said processor of said at least one medical device;
emitting a plurality of signals to a plurality of other medical devices and/or to an external processor, said plurality of signals which provide said anatomic and positional data to said plurality of other medical devices and/or to said external processor;
wherein said at least one processor dynamically analyzes said anatomic and positional data on a continuous basis such that said at least one medical device at least partially autonomously navigates to a desired position in the patient.
